(19) United States
(12) Patent Application Publication
(10) Pub. No.: US 2023/0357388 A1
(43) Pub. Date: Nov. 9, 2023

(54) IMMUNOTHERAPY

(71) Applicant: THE FRANCIS CRICK INSTITUTE LIMITED, London, Greater London (GB)

(72) Inventors: Caetano REIS E SOUSA, London, Greater London (GB); Evangelos GIAMPAZOLIAS, London, Greater London (GB); Oliver SCHULZ, London, Greater London (GB); Naren SRINIVASAN, London, Greater London (GB); Oliver GORDON, London, Greater London (GB); Probir CHAKRAVARTY, London, Greater London (GB)

(21) Appl. No.: 18/028,658

(22) PCT Filed: Sep. 24, 2021
(86) PCT No.: PCT/EP2021/076412

§ 371 Date: Mar. 27, 2023

(30) Foreign Application Priority Data
Sep. 25, 2020 (GB) 2015190.8
Sep. 25, 2020 (GR) 20200100581

(51) Int. Cl. C07K 16/28; A61K 45/06; A61P 37/02; G16H 50/20; C12Q 1/6886

(52) U.S. Cl. CPC C07K 16/28; A61K 45/06; A61P 37/02; G16H 50/20; C12Q 1/6886

(57) ABSTRACT

Gelsolin inhibitors are provided for use in treating diseases such as infectious diseases and cancer. Prognostic methods and methods for screening for gelsolin inhibitors are also provided.

### FIELD OF THE INVENTION

The present invention relates to immunotherapies for cancers and other diseases. In particular, the invention relates to immunotherapies that enhance antigen presentation of disease-related antigens. Diagnostic and screening methods are also provided.

### BACKGROUND

Cross-presentation (XP) refers to a process, performed by antigen-presenting cells (APCs), of presenting exogenous antigens on MHC class I molecules to CD8+ T cells, which can become cytotoxic T lymphocytes (CTL). XP by type 1 conventional dendritic cells (cDC1) is critical for priming anti-cancer CD8+ T cells. In both mice and humans, cDC1 express high levels of DNGR-1 (a.k.a., CLEC9A), a receptor that binds to actin filaments (F-actin) exposed by dead cell corpses^{1,2}. Binding DNGR-1 triggers XP of the antigens associated with the corpses (although the intracellular signalling events and mechanisms underlying XP in general have remained poorly understood). Some actin binding proteins (ABPs) such as myosin II potentiate DNGR-1 triggering by cross-linking the actin filaments to increase their avidity as DNGR-1 agonists³.

F-actin (the fibrous, polymeric form of actin) is present at high concentrations in most cells. When released into systemic circulation following cell death or injury, F-actin causes adverse pathophysiologic consequences, such as increased blood viscosity and disturbances in microvascular flow, activation of platelets with resulting platelet aggregation, microvascular thrombosis and release of proinflammatory mediators. F-actin exposure can therefore lead to secondary tissue damage due to this toxicity.

Gelsolin (GSN) is a multifunctional protein which can act to sever, cap and nucleate actin filaments. It is expressed both in the extracellular fluids and in the cytoplasm of a majority of human cells, and it is implicated in a variety of both physiological and pathological processes. Both cytoplasmic and secreted forms are coded by the same gene, with the secreted form (sGSN) being slightly longer. The field considers skeletal, cardiac and smooth muscles to be the main sources of secreted gelsolin in the bloodstream. sGSN can also be found in other fluids such as the lymphatic and CSF.

sGSN is one of two abundant ABPs that are present in the serum and plasma of all mammals, the other being Gc globulin. These ABPs are thought to form part of an actin-scavenging system, which contributes to the removal of potentially pathological actin filaments released by dying cells following tissue damage. In this system, sGSN binds to F-actin in a Ca²⁺ dependent manner and severs the filaments for subsequent depolymerisation, which is facilitated by Ca²⁺ independent sequestering of monomeric G-actin by Gc globulin.

### SUMMARY OF THE INVENTION

Although DNGR-1 activation is potentiated by some ABPs, the inventors wondered whether other ABPs might instead inhibit DNGR-1 activation. The inventors found that sGSN competitively blocks DNGR-1 binding to ligand and decreases cross-presentation (XP) of dead cell-associated antigens by cDC1 in vitro. More particularly, sGSN outcompetes DNGR-1 for binding to F-actin rather than simply cause loss of the ligand through filament severing. The inventors also found that sGSN deficient mice exhibit increased DNGR-1-dependent and CD8⁺ T cell-dependent resistance to challenge with a variety of transplantable tumours and display greater responsiveness to immunotherapy with checkpoint inhibitors. Moreover, in humans, the inventors found that lower levels of sGSN encoding transcripts in the tumour microenvironment (TME) are associated with increased patient survival rates in several cancer settings, suggesting a role of sGSN in cancer immunoevasion. These results identify sGSN as a natural in vivo barrier to XP of tumour antigens and priming of anti-cancer CD8+ T cell responses that could be exploited therapeutically (FIG. 13). It further indicates that inhibition of anti-cancer immunity can stem from circulating sGSN in plasma and/or local sGSN made in the TME. Thus, at its broadest, the invention relates to the inhibition of sGSN to promote XP; or the use of sGSN to reduce XP. This finds important applications in the context of cancer therapies and in the treatment of infectious diseases (by promoting XP) or therapy for autoimmune conditions (by reducing XP).

Accordingly, in one aspect, the invention provides a method of treating a disease in a subject, the method comprising administering a gelsolin inhibitor to the subject, wherein the gelsolin inhibitor inhibits gelsolin from binding F-actin. In a related aspect, the invention provides a gelsolin inhibitor for use in a method of treating a disease in a subject, the method comprising administering the gelsolin inhibitor to the subject, wherein the gelsolin inhibitor inhibits gelsolin (e.g., sGSN) from binding F-actin. The gelsolin may be sGSN and/or gelsolin that has been released into systemic circulation from a ruptured cell, e.g. a cell in the tumour microenvironment such as a ruptured cancer cell. Thus, the gelsolin inhibitor may be termed “sGSN inhibitor”. The disease may be a cancer, or an infectious disease. As described herein, by inhibiting sGSN from binding F-actin, the invention promotes cross-presentation (XP) of disease-related antigens by cDC1 (FIG. 13). The patient's immune response is thus enhanced. The sGSN inhibitor may inhibit sGSN from binding F-actin through a binding interaction between the sGSN inhibitor and sGSN, or through a binding interaction between the sGSN inhibitor and F-actin, or through a reduction in sGSN expression caused by the sGSN inhibitor. Preferably, sGSN inhibitor binds to sGSN itself. For instance, the gelsolin inhibitor may be an anti-gelsolin antibody or a gelsolin-binding aptamer. Alternatively, the sGSN inhibitor may be an RNAi molecule, such as an siRNA that reduces sGSN expression. In some embodiments, the interaction between sGSN and F-actin is measured using a dot blot assay as described herein. In some embodiments, the F-actin is dead cell-associated F-actin. In some embodiments, the F-actin is released into systemic circulation from a ruptured cell. In some embodiments, the gelsolin inhibitor binds gelsolin that has been released into systemic circulation from a ruptured cell.

In embodiments in which the disease is a cancer, e.g. a carcinoma, the cancer may be a liver cancer, e.g. a liver hepatocellular carcinoma (LIHC), a head and neck cancer e.g. a head and neck squamous cell carcinoma (HNSC), a glioma, e.g. a low grade glioma (LGG), or a gastric cancer, e.g. a stomach adenocarcinoma (STAD). The cancer may express a neoantigen, for instance a neoantigen corresponding to a protein associated with the actin cytoskeleton. In other embodiments, the neoantigen results from mutations in proteins that associate with F-actin, that is, a mutation in an F-actin binding protein (FABP). The neoantigen may be a mutated FABP. In embodiments in which the disease is a cancer, the treatment may comprise the administration of another cancer therapy in addition to the sGSN inhibitor. For instance, the treatment may also comprise the administration of an additional immunotherapy such as a checkpoint inhibitor to the patient. The checkpoint inhibitor may be an antibody that binds PD-1, PD-L1, CTLA4, TIM3, KIR, LAG3, or VISTA. Additionally or alternatively, the treatment may also comprise the administration of a cytotoxic agent. Additionally or alternatively, the treatment may also comprise the administration of a radiotherapy. Preferably, radiotherapy is administered before, or shortly after sGSN inhibitor administration, to cause cancer cell death thus exposing cancer antigens for XP by the antigen presenting cells. Additionally or alternatively, the treatment may also comprise a surgical procedure to remove at least part of the cancer. The surgical procedure may take place before sGSN inhibitor administration.

The sGSN inhibitor may be administered to the patient via injection, for instance intravenous injection or intratumoural injection. In embodiments in which the treatment comprises administration of an additional immunotherapy or cytotoxic agent, these may be administered via injection. The sGSN inhibitor, the additional immunotherapy and/or the cytotoxic agent may be co-administered, either in a single injection or in separate injections given at the same time (on the same day).

In embodiments in which the disease is an infectious disease, the disease may be a viral infection, a bacterial infection, or a parasitic infection. For instance, the disease may be caused by a viral, bacterial or parasitic pathogen that produces FABPs. Shigella and Listeria are exemplary pathogens that produce FABPs.

In another aspect, the invention provides a method of prognosing cancer patients. A cancer patient can be categorised as having a good prognosis or a bad prognosis by measuring the transcript and/or protein expression level of sGSN in a sample that has been taken from the patient, and comparing the sGSN transcript and/or protein expression level against a reference value which is the average level of sGSN transcript and/or protein expression in samples for patients of the same age, sex and disease stage, wherein, if the cancer patient has a lower level of sGSN transcript and/or protein expression than the reference value, the patient is categorised as having a good prognosis, and if the cancer patient has a higher level of sGSN transcript and/or protein expression than the reference value, the patient is categorised as having a bad prognosis. The sample may be from a cancer biopsy. Alternatively, the sample may be a plasma or blood sample.

The method may further comprise measuring the transcript and/or protein expression level of DNGR-1 and/or myosin II in the sample, and comparing these marker transcript and/or protein expression levels against a reference value or reference values which is/are the average level of the respective marker in samples from a population of other cancer patients of the same age, sex and disease stage. The reference value or values may be determined using data from The Cancer Genome Atlas (TCGA). The cancer may express a neoantigen, for instance a neoantigen corresponding to a protein associated with the actin cytoskeleton. In some embodiments, the neoantigen results from mutations in proteins that associate with F-actin, that is, a mutation in an F-actin binding protein (FABP). The neoantigen may be a mutated FABP.

Following prognosis, the cancer patient may be treated with one of the therapies disclosed herein.

In another aspect, the invention provides a method of screening for an immunotherapy agent, the method comprising providing a homogenous population of reporter cells that expresses DNGR-1 and then:
a) contacting one of the reporter cells with F-actin under conditions suitable to detect DNGR-1 signalling to establish a reference level;
b) contacting another of the reporter cells with F-actin in the presence of sGSN under the same conditions as in a), wherein sGSN is present at a concentration sufficient to substantially reduce the level of DNGR-1 signalling; and
c) contacting yet another of the reporter cells with F-actin in the presence of both sGSN and a candidate agent, wherein the conditions and sGSN concentration is the same as in b)
   wherein, if the level of DNGR-1 signalling in c) is greater than the level of DNGR-1 signalling in b), then the candidate agent is selected as an immunotherapy agent.

In a further aspect, the invention provides a method of treating an autoimmune disease in a subject, the method comprising increasing the level of gelsolin (e.g., sGSN) in the subject. In a related aspect, the invention provides gelsolin (e.g., sGSN) for use in a method of treating an autoimmune disease in a subject, the method comprising increasing the level of gelsolin (e.g., sGSN) in the subject. The level of gelsolin (e.g., sGSN) can be increased by administering gelsolin (e.g., sGSN) or by administering a gene therapy that increases gelsolin (e.g., sGSN) expression. The increased gelsolin (e.g., sGSN) levels can be targeted to the part of the subject that exhibits the autoimmune reaction (for instance the joints in rheumatoid arthritis, or the gut in inflammatory bowel disease).

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. sGSN inhibits DNGR-1 binding to F-actin. Flow cytometric analysis of bead-bound F-actin treated or not with 10 mg/mL sGSN before staining with DNGR-1 ECD, anti-GSN, or anti-actin antibodies. Numbers above graphs represent mean fluorescence intensity for each of the three samples. Black plots represent control beads; dark grey plots represent F-actin beads; light grey plots represent F-actin beads+sGSN.

FIG. 2. Serum from mice lacking sGsn no longer inhibits DNGR-1 binding to F-actin. Dot blot analysis of DNGR-1 ECD binding to immobilized F-actin, pre-treated or not with FCS or 10% mouse serum from WT or sGsn-deficient mice. #1 and #2 represent serum from individual mice.

FIG. 3. Loss of sGsn increases cancer control in mice. Growth profile of 0.2×10⁶ 5555 Braf^{v600E} cancer cells implanted in WT littermate control (sGsn^{+/+}) mice (n=5) and sGsn^{-/-} mice (n=5).

FIG. 4. Preferential control in sGsn^{-/-} mice of tumours bearing model antigens engineered to bind F-actin. Growth profile following subcutaneous inoculation of cancer cell lines expressing LA-OVA-mCherry into WT (C57BL/6J) or sGsn^{-/-} mice. 0.5×10⁶ MCA-205 LA-OVA-mCherry cancer cells implanted in WT (n=10) or sGsn^{-/-} (n=10) mice (left-hand panel). 0.3×10⁶ B16F10 LA-OVA-mCherry cancer cells implanted in WT (n=10) or sGsn^{-/-} (n=10) mice (right-hand panel).

FIG. 5. Loss of sGsn in mice increases responsiveness to cancer immunotherapy. Growth profile following subcutaneous inoculation of cancer cell lines expressing LA-OVA-mCherry into WT (C57BL/6J) or sGsn^{-/-} mice. 0.3×10⁶ B16F10 LA-OVA-mCherry cancer cells implanted in WT or sGsn^{-/-} mice that received 200 mg of isotype control or anti-PD-1 monoclonal antibody intraperitoneally (i.p.) every 3 days from day 3 to day 14. WT+isotype (n=10), sGsn^{-/-}+isotype (n=9), WT+anti-PD-1 (n=10), sGsn^{-/-}+anti-PD-1 (n=10) (left-hand panel). Growth profile of 0.5×10⁶ MCA-205 cancer cells implanted in WT or sGsn^{-/-} mice. Mice received 50 mg of Poly(I:C) or PBS (days 7 and 11) injected intratumorally in the presence of 50 mg of isotype control or anti-CTLA-4 (days 6 and 12) injected i.p. WT+PBS+isotype (n=6 mice), sGsn^{-/-}+PBS+isotype (n=5 mice), WT+Poly(I:C)+anti-CTLA-4 (n=8 mice), sGsn^{-/-}+Poly(I:C)+anti-CTLA-4 (n=8 mice) (right-hand panel).

FIG. 6. Expression of sGSN in mouse tumours allows immune escape in sGsn−/− mice. Growth profile of 0.5×10⁶ MCA-205 LA-OVA-mCherry cancer cells expressing either cGSN or sGSN, implanted in WT (n=9, cGSN, n=9, sGSN) or sGsn^{-/-} mice (n=7, cGSN, n=8, sGSN).

FIG. 7. Low expression of sGSN in liver, head & neck, and stomach cancer correlates with increased overall survival. Prognostic value of sGSN transcript levels for overall survival comparing samples with lowest (sGSN^{Low}) and highest (sGSN^{High}) expression in the indicated TCGA datasets. Liver hepatocellular carcinoma (LIH), bottom (n=74) and top (n=74) 20% of patient cohort. Head and neck squamous cell carcinoma (HNSC), bottom (n=104) and top (n=104) 20% of patient cohort. Stomach adenocarcinoma (STAD), bottom (n=41) and top (n=41) 10% of patient cohort.

FIG. 8. Stomach cancer: high CLEC9A expression correlates with survival in the sGSNLow subgroup of stomach cancer. Prognostic value of CLEC9A expression for cancer patient overall survival comparing top and bottom quartiles of sGSN^{Low} and sGSN^{High} subgroups in the indicated TCGA dataset.

FIG. 9. Tumour exome analysis: mutation prevalence in F-actin binding proteins (FABPs). Pevalence (percentage of tumors withRl mutation in the indicated class of genes) of mutation in F-actin-binding proteins in the indicated TCGA datasets.

FIG. 10. Lower intratumoural sGSN mRNA expression correlates with survival in patients with tumours bearing mutations in genes encoding F-actin binding proteins (FABPs). Prognostic value of sGSN transcript levels for overall survival comparing samples with lowest (sGSN^{Low}) and highest (sGSN^{High}) expression in the presence (Pos) or absence (Neg) of tumor mutational burden in F-actin-binding proteins (FABPs) in the indicated TCGA datasets.

FIG. 11. Lower intratumoural sGSN mRNA expression does not correlate with survival in patients with tumours lacking mutations in genes encoding FABPs. Prognostic value of sGSN transcript levels for overall survival comparing samples with lowest (sGSN^{Low}) and highest (sGSN^{High}) expression in the presence (Pos) or absence (Neg) of tumor mutational burden in F-actin-binding proteins (FABPs) in the indicated TCGA datasets.

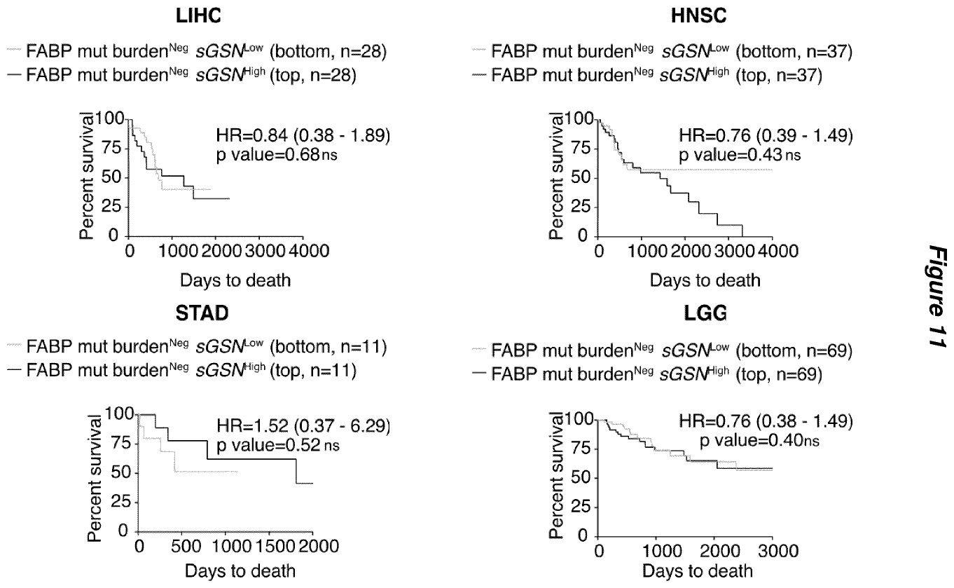

FIG. 12. Lower intratumoural sGSN mRNA expression does not correlate with survival in patients with tumours bearing mutations in genes encoding microtubule-binding proteins (MBPs). Prognostic value of sGSN transcript levels for overall survival comparing samples with lowest (sGSN^{Low}) and highest (sGSN^{High}) expression in the presence (Pos) or absence (Neg) of tumor mutational burden in microtubule-binding proteins (MBPs) for cancer patient overall survivalin the indicated TCGA datasets.

FIG. 13. Schematic of sGSN involvement in cancer immune evasion. sGSN in the TME promotes cancer immune evasion by inhibiting F-actin binding to DNGR-1, thus, leading to impairment of phagosomal rupture in cDC1 and subsequent cross-presentation, preferentially of neoantigens associated with actin cytoskeleton.

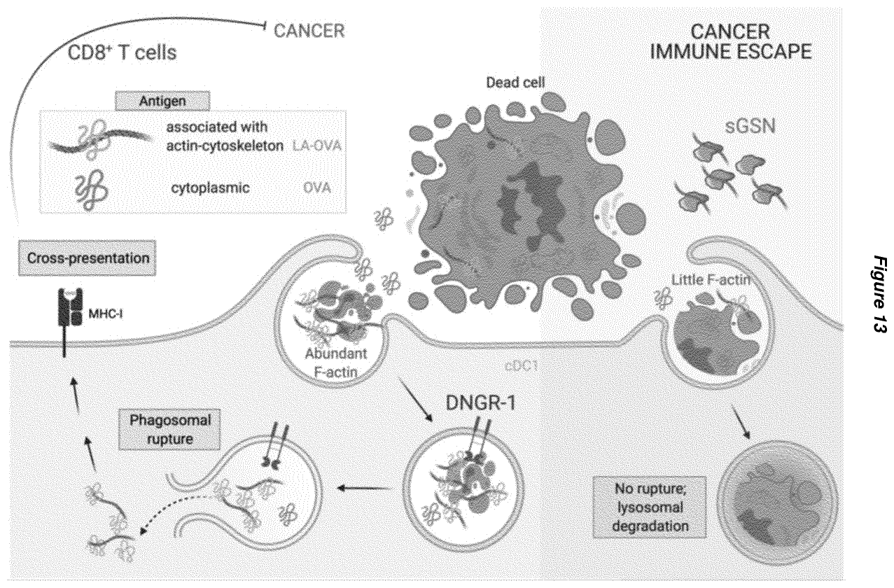

### DETAILED DESCRIPTION OF THE INVENTION

Cross-presentation (XP) of tumour antigens by type 1 conventional dendritic cells (cDC1) is critical for priming anti-cancer CD8+ T cells. As such, promoting cDC1 XP may enhance the ability of the immune system to control cancer and might overcome patient unresponsiveness to checkpoint blockade immunotherapy. In both mice and humans, cDC1 express high levels of DNGR-1 (a.k.a., CLEC9A), a receptor that binds to F-actin exposed by dying cells and signals to promote XP of antigens associated with the corpses. However, actin exposed by dead cells may be rapidly targeted for elimination by the plasma actin-scavenging system, which would antagonise DNGR-1-mediated recognition of dying tumour cells and dampen anti-cancer immunity. Consistent with this hypothesis, here the inventors show that secreted gelsolin (sGSN), the component of the plasma actin-scavenging system that specifically binds to and severs F-actin, competitively blocks DNGR-1 binding to ligand and decreases cross-presentation of dead cell-associated antigens by cDC1. Mice selectively deficient in sGSN display increased DNGR-1-dependent resistance to challenge with a variety of transplantable tumours, especially ones expressing neoantigens associated with the actin cytoskeleton, and exhibit greater responsiveness to immunotherapy with checkpoint blockade agents. Increased resistance of sGSN knock-out (sGsn^{-/-}) mice to tumours is attributable to increased DNGR-1-dependent cross-priming as it is abrogated by depletion of CD8+ T cells or by crossing sGsn^{-/-} mice to mice deficient in DNGR-1. In humans, lower levels of transcripts encoding sGSN in the tumour microenvironment, as well as high frequency of mutations in proteins associated with the actin cytoskeleton, are associated with increased patient survival in several cancers. These results, which are discussed in more detail in the Examples section below, reveal a natural in vivo barrier to cross-presentation of tumour antigens that dampens anti-cancer CD8+ T cell responses and could be exploited therapeutically.

sGSN Inhibitors

The sGSN inhibitors envisaged herein reduce the extent of binding between sGSN and F-actin. In some embodiments, the sGSN inhibitors are antibodies, for instance anti-sGSN antibodies. Preferably, the antibody is a monoclonal antibody. (As explained in the section below, the term antibody herein encompasses antigen-binding fragments thereof.) Therefore, it will be understood that in some embodiments the gelsolin inhibitor does not cross the plasma membrane of a cell.

Aptamers are short DNA/RNA/peptide molecules that can bind specifically to a target molecule. Aptamers specific for a particular target are often selected from a large pool of randomly generated libraries of molecules, e.g. by using the Systematic Evolution of Ligands by Exponential Enrichment (SELEX) method⁴⁴. The SELEX method involves exposing a random sequence library to a specific target and amplifying the bound molecules which are then subjected to additional rounds of selection. After multiple rounds of selection, specific aptamers identified for binding to the target molecule (i.e. sGSN or F-actin) can be subjected to further rounds of modifications to improve their binding affinity and stability. Aptamers can be readily conjugated to additional nucleic acid moieties and/or additional aptamer moieties, thus facilitating enhanced, multimeric and/or multi-specific binding.

Besides antibodies and aptamers, other sGSN-binding agents and F-actin-binding agents, such as peptides and small molecules are envisaged. The inhibitory action of sGSN-binding agents can be assessed by using the methods described herein. For instance, the ability of a sGSN-binding agent to inhibit sGSN from binding F-actin immobilised on microspheres or on nitrocellulose membranes (discussed in the methods part of the Examples section below) can be confirmed. The ability of sGSN-binding agents to prevent sGSN from inhibiting the agonistic activity of F-actin on DNGR-1 can be confirmed using the DNGR-1 signalling reporter cell lines discussed in the methods part of the Examples section below.

In contrast to the antibodies, aptamers, peptides and small molecules discussed herein, which act as sGSN inhibitors by binding sGSN molecules, small RNA molecules may be employed to inhibit sGSN function by down-regulating sGSN expression. Collectively, described as RNAi-based inhibitors of sGSN expression, these include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing. In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences. The ability of an RNAi to reduce sGSN expression levels can be determined by routine testing and screening methods.

Antibody-Based sGSN Inhibitors

Antibodies that bind gelsolin are widely available. Preferably, the antibody is a monoclonal antibody. For instance, monoclonal antibodies derived from a variety of mammalian species that specifically bind to human gelsolin are available from AbCam (see product codes ab109014, ab75832, ab214342, ab11081, ab134183, ab236029, ab247406, ab247406 and ab225096 for instance). Similar ranges of anti-gelsolin antibodies are available from other suppliers such as ThermoFisher and Santa Cruz Biotechnology (SCBT). In view of today's techniques in relation to monoclonal antibody technology, further anti-sGSN antibodies can be readily prepared.

Anti-F-actin antibodies are also known, and are commercially available e.g. from AbCam; see product codes ab205, ab130935, ab272559, ab83746, ab140435 as just five examples. Thus, the sGSN inhibitor may be an anti-F-actin antibody that prevents sGSN but not DNGR-1 binding.

The antibody may be a target-binding fragment of an antibody (for example a Fab fragment) or a synthetic antibody fragment (for example a single chain Fv fragment [ScFv] or single-domain antibody/nanobody). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen. Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel D L (2002). “Recombinant monoclonal antibody technology”. Schmitz U, Versmold A, Kaufmann P, Frank H G (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799)).

Polyclonal antibodies are useful in the methods of the invention. Monospecific polyclonal antibodies are preferred. Suitable polyclonal antibodies can be prepared using methods well known in the art.

Fragments of antibodies, such as Fab and Fab2 fragments may also be used as can genetically engineered antibodies and antibody fragments. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sd. USA 85, 5879) and single domain antibodies (sdAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are covalently linked, e.g. directly, by a peptide or by a flexible oligopeptide. Fab, Fv, ScFv and sdAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and dAb fragments are monovalent, having only one antigen combining site. Synthetic antibodies which bind to a target discussed herein may also be made using phage display technology as is well known in the art (e.g. see “Phage display: a molecular tool for the generation of antibodies—a review”. Placenta. 21 Suppl A: S106-12. Helen E. Chadd and Steven M. Chamow; “Phage antibodies: filamentous phage displaying antibody variable domains”. Nature 348 (6301): 552-554).

RNAi Based sGSN Inhibitors

RNAi-based inhibitors of sGSN expression can be used as the sGSN inhibitors in the context of this invention. In some embodiments, the RNAi is an siRNA that inhibits sGSN expression. The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed on John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such as Ambion's siRNA finder, see http://www.ambion.com/techlib/misc/siRNA_finder.html. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of the sGSN mRNA. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of sGSN mRNA.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

### Vectors

A “vector” as used herein is an oligonucleotide molecule (DNA or RNA) used as a vehicle to transfer foreign genetic material into a cell. The vector may be an expression vector for expression of the foreign genetic material in the cell. Such vectors may include a promoter sequence operably linked to the nucleotide sequence encoding the gene sequence to be expressed. A vector may also include a termination codon and expression enhancers. Any suitable vectors, promoters, enhancers and termination codons known in the art may be used to express the chimeric receptor of the invention in a cell or tissue.

The therapy vector can be used to introduce a nucleic acid encoding an RNAi based sGSN inhibitor into a recipient cell or tissue. The vector can be a gene therapy vector. In some embodiments, the gene therapy vector is a viral vector. The viral vector may be an adenoviral vector, an AAV or a lentiviral vector. For some applications, it is advantageous to use a viral vector that is pseudotyped with an envelope protein that facilitates the transduction of hematopoietic stem cells and/or progenitor cells. In some embodiments, the nucleic acid is introduced into the mammalian cell using the CRISPR-CAS9 system.

### Pharmaceutical Compositions

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. the vectors, cells and or chimeric receptors, used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

### Routes of Administration

Medicaments and pharmaceutical compositions according to aspects of the present invention may be formulated for administration by a number of routes, including but not limited to, parenteral, intravenous, intra-arterial, intramuscular, intratumoural, oral and nasal. The medicaments and compositions may be formulated in fluid or solid form. Fluid formulations may be formulated for administration by injection to a selected region of the human or animal body.

Combinations with Other Anticancer Treatments

As described herein, the medical methods, medical uses and pharmaceutical compositions of the invention may involve the sGSN inhibitor in combination with another anticancer treatment. In some embodiments, the anticancer treatment is an additional immunotherapy.

Currently, the most common cancer immunotherapies are checkpoint inhibitors. The sGSN inhibitor of the invention may be used in combination with a checkpoint inhibitor. Checkpoint inhibitors suitable for use in combination with the sGSN inhibitor of the invention includes a checkpoint inhibitor that inhibits CTLA4, cytotoxic T-lymphocyte-associated antigen 4; e.g. anti-CTLA4; anti-LAG3, lymphocyte activation gene 3; anti-PD1, programmed cell death protein 1 (eg, KEYTRUDA); PDL, anti-PD1 ligand; anti-TIM3, T cell membrane protein 3, anti-CD40L, anti-A2aR, adenosine A2a receptor; anti-B7RP1, B7-related protein 1; anti-BTLA, B and T lymphocyte attenuator; anti-GAL9, galectin 9; anti-HVEM, herpesvirus entry mediator; anti-ICOS, inducible T cell co-stimulator; anti-IL, interleukin; anti-KIR, killer cell immunoglobulin-like receptor; anti-LAG3, lymphocyte activation gene 3; anti-VISTA, V domain Ig Suppressor of T cell Activation; anti-B7-H3; anti-B7-H4; anti-TGFβ, transforming growth factor-β; anti-TIM3, T cell membrane protein 3; or anti-CD27.

Other immunotherapies, such as T cell therapy, can be used in conjunction with the sGSN inhibitors disclosed herein. T cell therapies include administration of autologous or allogeneic T cells. In some embodiments, the sGSN inhibitor is administered in combination with a CAR-T cell (a T cell that expresses a chimeric antigen receptor).

In some embodiments, the anticancer treatment is a cytotoxic chemotherapeutic, meaning that the sGSN inhibitor of the invention may be used in combination with a cytotoxic chemotherapeutic. Cytotoxic chemotherapeutic agents non-exclusively relates to alkylating agents, anti-metabolites, plant alkaloids, topoisomerase inhibitors, antineoplastics and arsenic trioxide, carmustine, fludarabine, IDA ara-C, myalotang, GO, mustargen, cyclophosphamide, gemcitabine, bendamustine, total body irradiation, cytarabine, etoposide, melphalan, pentostatin and radiation.

In some embodiments, the anticancer treatment is radiotherapy. In some embodiments, the anticancer treatment is surgery.

### Subject

The subject to be treated may be any animal or human. The subject is preferably mammalian, more preferably human. The subject may be a non-human mammal, but is more preferably human. The subject may be male or female. The subject may be a patient. Therapeutic uses may be in human or animals (veterinary use).

### Cancers

A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor. In some embodiments, the cancer is not a prostate cancer.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

Some cancers cause solid tumours. Such solid tumours may be located in any tissue, for example the pancreas, lung, breast, uterus, stomach, kidney or testis. In contrast, cancers of the blood, such as leukaemias, may not cause solid tumours—and may be referred to as liquid tumours.

The cancer that is the subject of the treatments and medical uses of the present invention may be triple negative breast cancer. The cancer that is the subject of the treatments and medical uses of the present invention may be unresponsiveness to immunotherapy with checkpoint inhibitors.

The cancer that is the subject of the treatments and medical uses of the present invention may be selected from the lists provided above. In some embodiments, the cancer is a liver cancer, a head and neck cancer, or a gastric cancer.

***

Aspects and embodiments of the present invention are discussed. Further aspects and embodiments will be apparent to those skilled in the art. All documents mentioned in this text are incorporated herein by reference.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### Exemplary Embodiments

1. A method of treating a cancer in a subject, the method comprising administering a gelsolin inhibitor to the subject to enhance an anticancer immune response.
2. A gelsolin inhibitor for use in a method of treating cancer, the method comprising administering the gelsolin inhibitor to the subject to enhance an anticancer immune response.
3. The method according to item 1, or the gelsolin inhibitor for the use according to item 2, wherein the gelsolin inhibitor is an anti-gelsolin antibody.
4. The method, or the gelsolin inhibitor for the use, according to any one of the preceding items, wherein the treatment comprises the administration of a checkpoint inhibitor to the patient.
5. The method, or the gelsolin inhibitor for the use, according to item 4, wherein the checkpoint inhibitor is selected from an anti-PD-1, anti-PD-L1, anti-CTLA4, anti-TIM3, anti-KIR, anti-LAG3, and an anti-VISTA antibody.
6. The method, or the gelsolin inhibitor for the use, according to any one of the preceding items, wherein the cancer is a liver cancer, a head and neck cancer, a glioma, or a gastric cancer.
7. The method, or the gelsolin inhibitor for the use, according to any one of the preceding items, wherein the treatment comprises the administration of radiotherapy.
8. The method, or the gelsolin inhibitor for the use, according to any one of the preceding items, wherein the cancer expresses a neoantigen.
9. The method according to item 8, wherein the neoantigen results from a mutation in an F-actin binding protein (FABP).
10. The method, or the gelsolin inhibitor for the use, according to any one of the preceding items, wherein the gelsolin inhibitor targets sGSN and/or gelsolin that has been released into systemic circulation from a ruptured cell in the tumour microenvironment.
11. The method, or the gelsolin inhibitor for the use, according to any one of the preceding items, wherein the F-actin is dead cell-associated F-actin.
12. The method, or the gelsolin inhibitor for the use, according to any one of items 1 to 10, wherein the F-actin is released into systemic circulation from a ruptured tumour cell.
13. A method of treating an infectious disease in a subject, the method comprising administering a gelsolin inhibitor to the subject to enhance an immune response against an antigen derived from a disease-causing agent selected from a viral particle, a bacterium, or a parasite.
14. A gelsolin inhibitor for use in a method of treating an infectious disease in a subject, the method comprising administering the gelsolin inhibitor to the subject to enhance an immune response against an antigen derived from a disease-causing agent selected from a viral particle, a bacterium, or a parasite.
15. A method of categorising a cancer patient as having a good prognosis or a bad prognosis, the method comprising measuring the transcript and/or protein expression level of sGSN in a sample that has been obtained from the patient, and comparing the sGSN transcript and/or protein expression level against a reference value which is the average level of sGSN transcript and/or protein expression in samples from a population of other cancer patients of the same disease stage, wherein, if the cancer patient has a lower level of sGSN transcript and/or protein expression than the reference value, the patient is categorised as having a good prognosis, and if the cancer patient has a higher level of sGSN transcript and/or protein expression than the reference value, the patient is categorised as having a bad prognosis.
16. The method according to item 15, wherein the method further comprises measuring the transcript and/or protein expression level of DNGR-1 and/or myosin II in the sample, and comparing these marker transcript and/or protein expression levels against a reference value or reference values which is/are the average level of the respective marker in samples from a population of other cancer patients of the same disease stage.
17. The method according to item 15 or item 16, wherein the reference value or values are determined using data from The Cancer Genome Atlas (TCGA).
18. The method according to any one of items 15 to 17, wherein the cancer expresses a neoantigen.
19. The method according to item 18, wherein the neoantigen results from a mutation in an F-actin binding protein (FABP).
20. A method of screening for an immunotherapy agent, the method comprising providing a homogenous population of reporter cells that expresses DNGR-1 and then:
   a) contacting one of the reporter cells with F-actin under conditions suitable to detect cross-presentation (XP), to establish a reference level of XP;
   b) contacting another of the reporter cells with F-actin in the presence of sGSN under the same conditions as in a), wherein sGSN is present at a concentration sufficient to substantially reduce the level of XP; and
   c) contacting yet another of the reporter cells with F-actin in the presence of both sGSN and a candidate agent, wherein the conditions and sGSN concentration is the same as in b)
   wherein, if the level of XP in c) is greater than the level of XP in b), then the candidate agent is selected as an immunotherapy agent.
21. Soluble gelsolin (sGSN) for use in a method of treating an autoimmune disease in a subject, the method comprising increasing the level of sGSN in the subject.

### EXAMPLES

### Secreted Gelsolin Inhibits 1 DNGR-1-Dependent Cross Presentation and Dampens Anti-Cancer Immunity

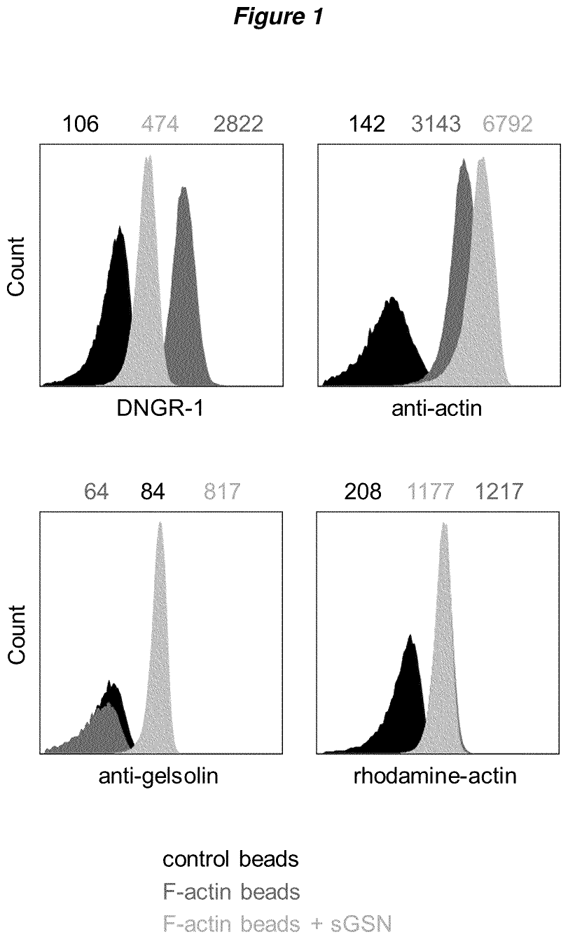

Although DNGR-1 activation is potentiated by some ABPs such as myosin II, the inventors wondered whether other ABPs might function instead as inhibitors of DNGR-1. The inventors noticed that foetal calf serum (FCS), used instead of milk powder as a blocking reagent in a dot blot with immobilised F-actin, inhibited binding of the extracellular domain of DNGR-1 (DNGR-1 ECD) in a dose dependent manner. To address if this involved actin-binding molecules present in FCS, the inventors mixed the serum with F-actin and discarded the latter, together with any bound material, by high-speed centrifugation. FCS treated in this manner failed to inhibit DNGR-1 binding to immobilised F-actin. Serum and plasma of all mammals contain two abundant ABPs, secreted gelsolin (sGSN) and Gc globulin, that together are thought to contribute to the removal of potentially pathological actin filaments released by dying cells following tissue damage^{4 -6}. In this, so-called, plasma actin-scavenging system, sGSN binds to F-actin in a Ca²⁺-dependent manner and severs the filaments for subsequent depolymerisation, which is facilitated by Ca²⁺-independent sequestering of monomeric G75 actin by Gc globulin⁷⁻¹¹. FCS inhibition of DNGR-1 binding to F-actin could be prevented by chelating Ca²⁺ ions, pointing to sGSN rather than Gc globulin as the serum ABP in question. Indeed, treatment of membrane-immobilised F-actin with human recombinant sGSN completely abolished binding of DNGR-1 while treatment with cofilin, a cellular ABP that also binds to and destabilises actin filaments^{12,13}, used as a control, had no effect. To quantitatively measure gelsolin interference with DNGR-1 binding, the inventors switched to flow cytometric analysis of bead-bound, fluorescent F-actin. Recapitulating the dot blot findings, binding of DNGR-1 ECD to F-actin beads was greatly reduced in the presence of sGSN (FIG. 1). The total amount of fluorescent rhodamine-actin on beads was unchanged by sGSN incubation and binding of anti-actin antibody was unaffected or even slightly increased, perhaps due to increased exposure of epitopes (FIG. 1). These results suggest that sGSN outcompetes DNGR-1 for binding to F-actin rather than simply cause loss of the ligand from beads through filament severing.

As expected, binding of sGSN to bead-bound F-actin and its ability to subsequently block DNGR-1 was prevented by calcium chelation. sGSN also decreased DNGR-1 binding to F-actin/myosin II complexes, although this was less pronounced than the effect on binding to naked F-actin. Thus, myosin II offers some degree of protection from sGSN, which may contribute to the potency of F-actin/myosin II complexes as DNGR-1 triggers³.

In vivo Studies

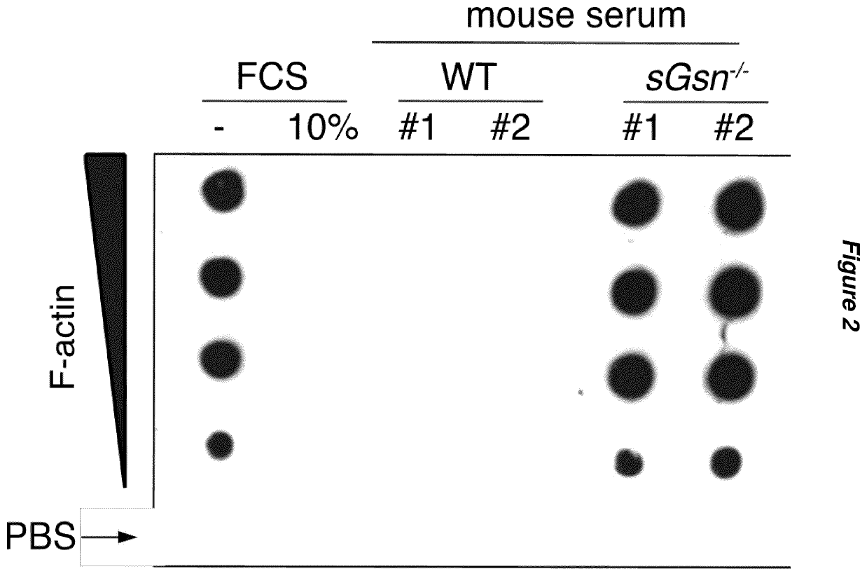

All cells make cytoplasmic GSN, which acts as an important regulator of actin filament disassembly^{14,15}. In contrast, sGSN is thought to be produced primarily by muscle cells¹⁶ using an alternatively-spliced exon in the Gsn gene that contains a start site and signal peptide ^{17,18}. By targeting that exon using CRISPR, the inventors generated C57BL/6 mice that lack plasma gelsolin (sGsn^{-/-}) but have intact cytoplasmic GSN. The inventors verified that sGsn^{-/-} mice develop and age normally, as expected from the fact that mice doubly deficient in cytoplasmic GSN and sGSN show only a mild phenotype in the C57BL/6 genetic background^{19,20}. sGsn^{-/-} mice had overall normal myeloid and lymphoid cell compartments in primary and secondary lymphoid organs and showed no signs of autoimmunity although they displayed marginally elevated levels of IgG and IgM auto-antibodies upon ageing (>1 year) Consistent with a normal immunological profile, sGsn^{-/-} mice displayed no impairment in their ability to resist bacterial (Streptococcus pneumoniae), parasite (Nippostrongylus brasiliensis) and viral (influenza A virus) challenges. The inventors compared serum from WT and sGsn^{-/-} mice for inhibition of DNGR-1 binding to F-actin. In the dot blot assay, binding of DNGR-1 to immobilised F-actin was blocked by pre-treatment of the membrane with serum from control mice, as with FCS, but not with serum from mice lacking sGSN (FIG. 2). The inventors also used serum from sGSN-deficient mice supplemented or not with a defined amount (10 μg/ml) of recombinant sGSN, a dose that is at least 10-fold lower than physiological levels of plasma sGSN (150-300 μg/ml) in a reporter assay of DNGR-1 triggering²¹. In the presence of sGSN, stimulation of the reporter cells with F-actin alone was undetectable up until a concentration of ligand (0.5 μM) that exceeded the amount of added sGSN (0.1 μM) by five-fold, suggesting that sGSN blocks DNGR-1 binding sites on F-actin in a stoichiometric manner. Using F-actin/myosin II as stimulus, DNGR-1 triggering was also affected by sGSN albeit to a lesser degree, consistent with the fact that myosin II could offer some degree of protection from sGSN inhibition. To assess the impact of sGSN on DNGR-1 triggering by dead cells, the inventors used UV-irradiated mouse embryonic fibroblasts or tumour cells s stimuli. Again, the inventors found robust inhibition of DNGR-1 triggering by dead cell corpses in the presence of sGSN. In contrast, the absence or presence of sGSN did not impact stimulation of reporter cells with plate-bound anti-DNGR-1 antibody, excluding non-specific effects. Unlike sGSN, the other component of the actin-scavenging system, Gc globulin, cannot bind to F-actin and is therefore unlikely to directly interfere with DNGR-1 triggering by ligand²²⁻²⁴. Consistent with that notion, sGSN-mediated inhibition of dead cell-induced stimulation of the reporter cells was similar whether the assay was carried out with serum from sGSN-deficient mice or serum from mice doubly deficient in sGSN and Gc globulin. Cytoplasmic gelsolin potentially released from dead cells was also not sufficient to interfere with DNGR-binding as the reporter cells were stimulated equally by killed cells from the parental (gelsolin-sufficient) 5555 Braf^{V600E} tumour cell line and from a stable 5555 Braf^{V600E} gelsolin knockdown (KD) line. This is likely a quantitative issue, as cytoplasmic gelsolin released from dead cells is rapidly diluted to below 1 μg/ml, the concentration required to inhibit DNGR-1 triggering. Finally, the inventors examined the effect of sGSN on cross presentation to OT-I T cells of dead cell-associated ovalbumin (OVA) antigen by the Mutu cDC1 cell line, which expresses DNGR-1. The OT-I response in cultures containing sGSN was significantly lower than that in sGSN-free mouse serum, indicating inhibition. In contrast, presentation of OVA (SIINFEKL)-peptide or cross-presentation of soluble OVA protein was not affected by sGSN, emphasising the specificity of the inhibitory effect of sGSN on cross-presentation of dead cell-associated antigen. The inventors concluded that sGSN is necessary and sufficient for inhibition of dead cell recognition by DNGR-1 and for decreasing cross-presentation of dead cell-associated antigens.

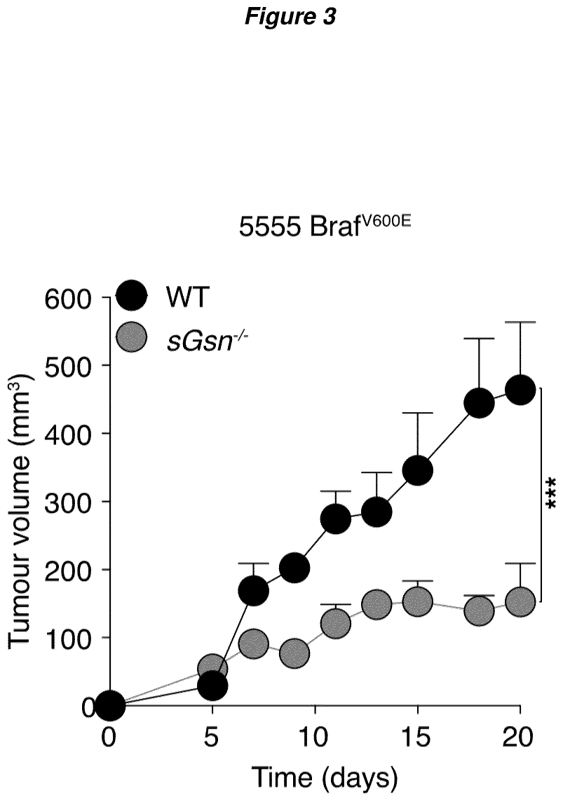

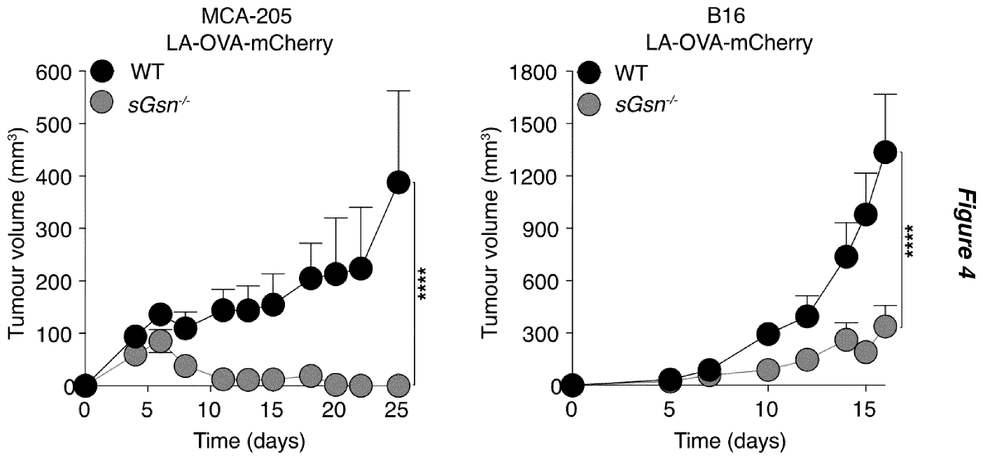

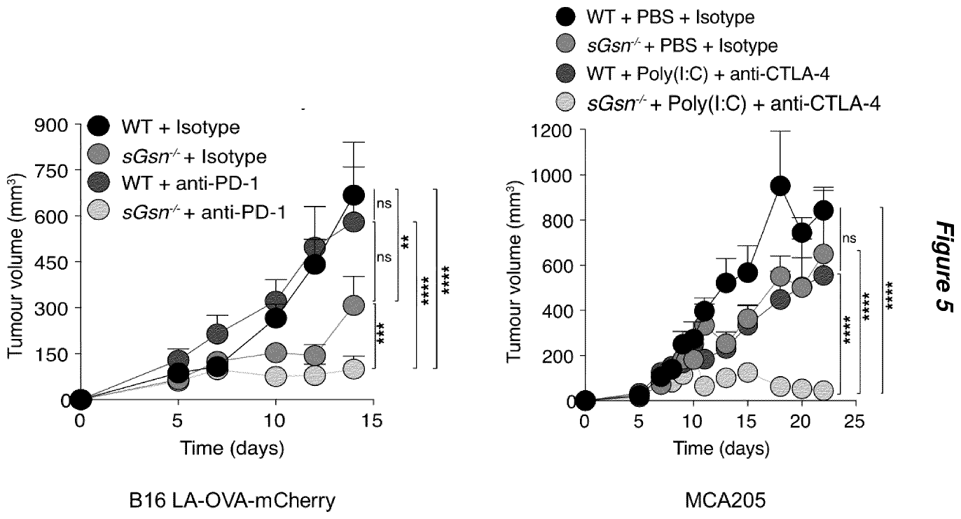

Cross-presentation of dead cell-associated antigens is thought to be a major mechanism utilised by cDC1 to prime anti-tumour CD8⁺ T cells²⁵⁻²⁸. As cross-presentation is a limiting factor in anti-tumour immunity, the inventors hypothesised that sGsn^{-/-} mice might display increased anti-tumour CD8+ T cell responses. Consistent with this hypothesis, highly immunogenic tumours derived from an OVA-expressing thymoma cell line (EG7), exhibited faster and increased rejection in sGSN-deficient mice compared to C57BL6 wildtype (WT) mice. To extend these findings and to test whether the relative tumour resistance of sGSN-deficient mice is more marked in settings in which the relevant tumour antigens are associated with the actin cytoskeleton²⁹, the inventors constructed a version of OVA lacking the signal sequence fused to the 17 amino acid sequence of the F-actin binding peptide LifeAct³⁰ (LA-OVA). The inventors further fused it to a fluorescent protein (mCherry) to allow antigen tracking and expressed the construct (LA-OVA-mCherry) in the weakly immunogenic fibrosarcoma cell line MCA-205. The inventors found that sGsn^{-/-} mice controlled LA-OVA-mCherry MCA-205 tumours much better than WT controls (FIG. 4). Indeed, complete tumour rejection accompanied by prolonged tumour remission was only seen in sGSN-deficient hosts. Similarly, expression of LA-OVA-mCherry in the poorly immunogenic B16F10 melanoma cell line permitted tumour control preferentially in the sGSN-deficient mouse strain. This was not the case with B16F10 expressing non-secreted OVA not fused to the LA peptide even though that cell line expressed higher levels of ovalbumin than the LA-OVA-transduced B16F10 line, which indicates that tumour control in sGsn^{-/-} mice correlates with cytoskeletal association of antigen rather than antigen levels. Control of LA-OVA-mCherry B16 tumours in sGsn^{-/-} mice was further enhanced by anti-PD-1 immune checkpoint blockade, which, by itself, had no effect on this particular cell line when implanted in WT mice (FIG. 5). The tumour resistance phenotype of sGSN-deficient mice could also be revealed in tumours that were not engineered to express any model tumour antigen such as the 5555 Braf^{V600E} melanoma cell line (FIG. 3) or even in the parental MCA-205 line not expressing OVA when its immunogenicity was increased by treating with the immune checkpoint inhibitor anti-CTLA-4 together with the immune stimulator poly(I:C) (FIG. 5). Thus, sGsn^{-/-} mice exhibit greater resistance to a variety of transplantable tumours, especially ones bearing tumour neoantigens that associate with the actin cytoskeleton. The fact that sGsn^{-/-} mice are more responsive to immunotherapy with checkpoint inhibitors suggests an immune-dependent underlying mechanism of resistance.

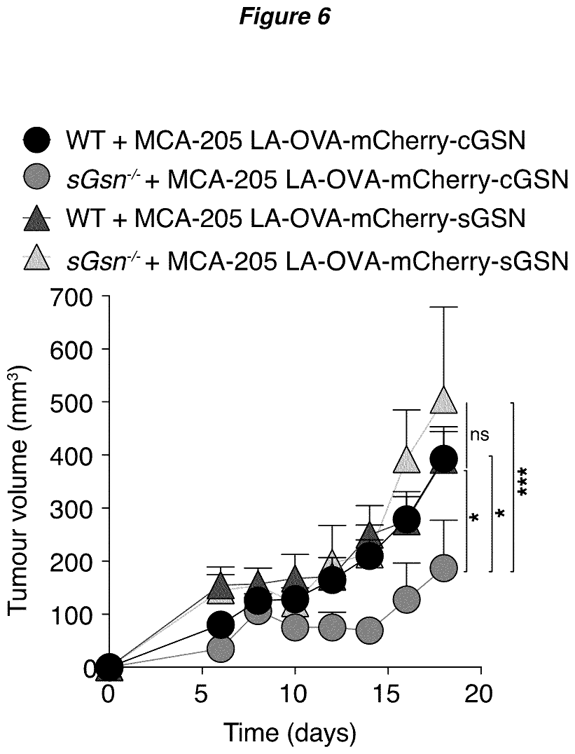

Consistent with the latter, the inventors found a higher number and frequency of intratumoral OVA-specific (pentamer+) CD8+ T cells in sGsn^{-/-} mice bearing B16-LA-OVA-mCherry tumours. This effector CD8+ T cell response against tumour antigens was responsible for the observed relative tumour resistance of sGsn^{-/-} mice as the latter was completely abrogated by antibody-mediated CD8+ T cell depletion. To test the involvement of DNGR-1 in enhanced tumour control in sGsn^{-/-} mice, the inventors generated mice lacking both DNGR-1 and sGsn (sGsn^{-/-}; Clec9a^{gfp/gfp}). Notably, additional loss of Clec9a completely reversed the tumour resistance of sGSN-deficient mice but did not impact tumour growth in sGSN-sufficient hosts. Collectively our data indicate a specialised role for DNGR-1 in controlling tumours in a sGSN-deficient background. Consistent with the notion that DNGR-1 is a dedicated receptor for cross-presentation of dead-cell associated antigens and does not impact cDC1 differentiation, migration and activation^{21,31}, sGSN deficiency had no impact on the accumulation of cDC1 in the tumour microenvironment or in the tumour draining lymph node. Notably, forced sGSN but not cGSN expression abrogated the relative resistance of sGsn^{-/-} mice to LA-OVA-mCherry tumors (FIG. 6), indicating that sGSN secretion by cancer cells can function as an escape strategy.

sGSN and Clec9a Transcription Levels in Human Cancer Patients

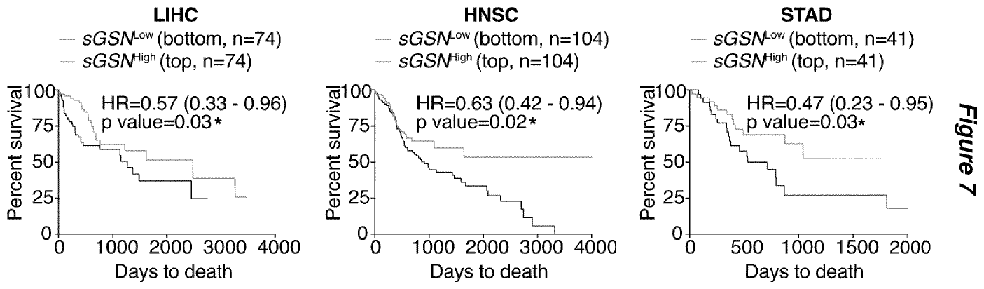

sGSN was found to be expressed in the vast majority of human tissues, accounting for more than half of total gelsolin transcript expression and it has been reported that human cancer cells can also secrete large amounts of sGSN, contributing to local extracellular concentrations up to 400 μg/ml. For instance, it has been recently reported that human prostate cancer cells and primary prostate tumours are also able to secrete sGSN, contributing to its local accumulation in the extracellular milieu³². The inventors hypothesized that production of sGSN in the tumour microenviroment by cancer and other cells could lead to elevated local levels of the protein irrespective of the amount circulating in plasma, impacting immunity and patient outcome. The inventors performed in silico analysis of gelsolin isoform expression data from The Cancer Genome Atlas (TCGA) and correlated them with patient survival. The dynamic range of sGSN transcript levels in TCGA datasets for liver hepatocellular carcinoma (LIHC, n=370), head and neck squamous cell carcinoma (HNSC, n=518) and stomach adenocarcinoma (STAD, n=408).Due to the limited dynamic range of sGSN transcript levels, optimal cut-offs were used to allow maximum segregation between the highest and lowest expressors while retaining enough data points for meaningful analysis. Analysis of all three tumour types revealed that lower sGSN transcript expression correlated positively with survival (FIG. 7), a difference that was not attributable to age, sex and disease stage. In line with these findings, expression of sGSN has been reported to be associated with poor clinical outcomes in ovarian and prostate cancer patients³². Of note, in our analysis, the expression of the cytoplasmic gelsolin isoform (cGSN) was not able to predict patient survival, highlighting a specific role for secreted but not cytoplasmic gelsolin in cancer progression The inventors further compared LIHC, HNSC and STAD tumours stratified by sGSN expression for differences in gene expression signatures using the REACTOME database. Low sGSN tumours showed specific enrichment for gene signatures of processes associated with MHC class I (cross)-presentation and cell death, frequently accompanied by gene signatures of adaptive immunity. Thus, as in mice, sGSN is associated with poorer cancer patient survival, likely by impairing immune-mediated control of tumours.

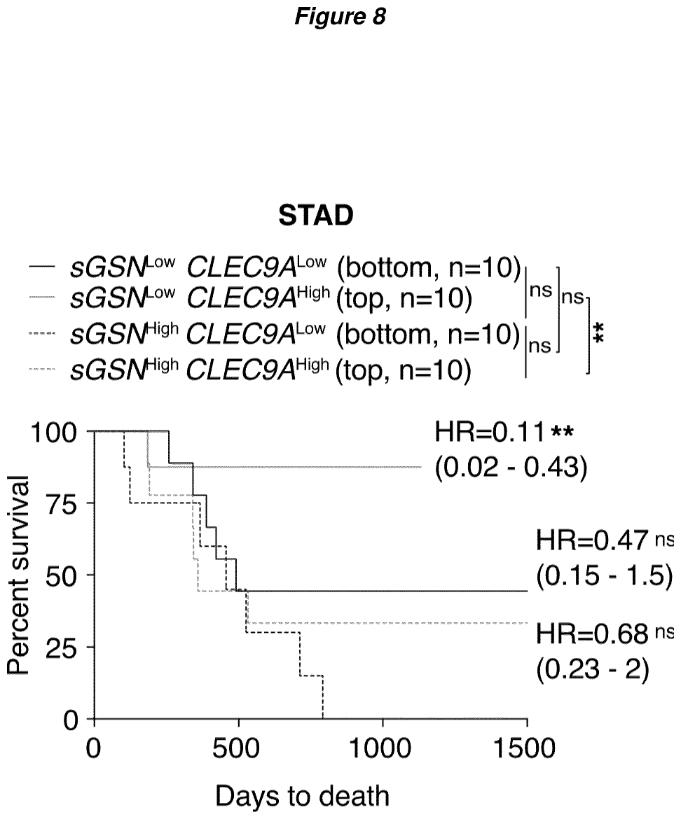

The inventors also determined the prognostic value of CLEC9A in overall cancer survival by comparing top and bottom patient quartiles. CLEC9A expression correlated positively with patient overall survival in LIHC and HNSC but not in the STAD dataset. CLEC9A expression correlated positively with patient overall survival in LIHC and HNSC but not in the STAD dataset (FIG. 8). The inventors found this to be the case and found it to be selective for sGSN as higher CLEC9A expression did not correlate with survival when patients were stratified on the basis of expression of cytoplasmic rather than secreted gelsolin. CLEC9A is a marker of cDC1 but a specific cDC1 gene signature³⁴ did not associate with STAD patient survival irrespective of sGSN expression levels, suggesting that the ability of CLEC9A to predict patient survival in the low sGSN patient group might predominantly reflect DNGR-1 receptor function rather than cDC1 content. In line with this possibility, both CLEC9A and “effector CD8 T cell” gene signature³⁴ correlated with “MHC class I (cross)-presentation related” gene signatures more strongly in the low sGSN than in the high sGSN subgroup of STAD patient. Furthermore, CLEC9A and “effector CD8 T cell” gene signatures also cross-correlated to a greater extent in the low sGSN subgroup when compared as part of “MHC class I (cross)-presentation related” gene signatures, highlighting their potential intersection in a common pathway. Importantly, by comparing the top and bottom quartiles as described before, the inventors found that, although “effector CD8 T cell” and “cross-presentation related” gene signatures did not on their own associate with survival in STAD patients, they were in conjuction, like CLEC9A expression, able to predict survival selectively in the low sGSN patient subgroup. Thus, as in mice, sGSN is associated with poorer cancer outcome, which correlates with lower CLEC9A-CD8 immune-mediated control.

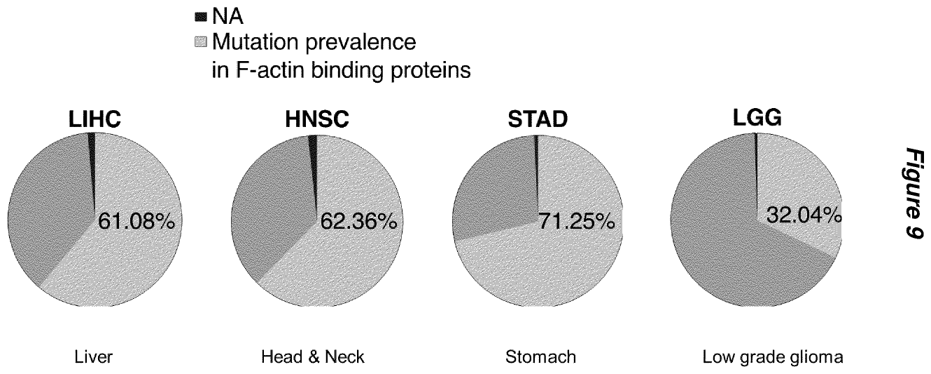

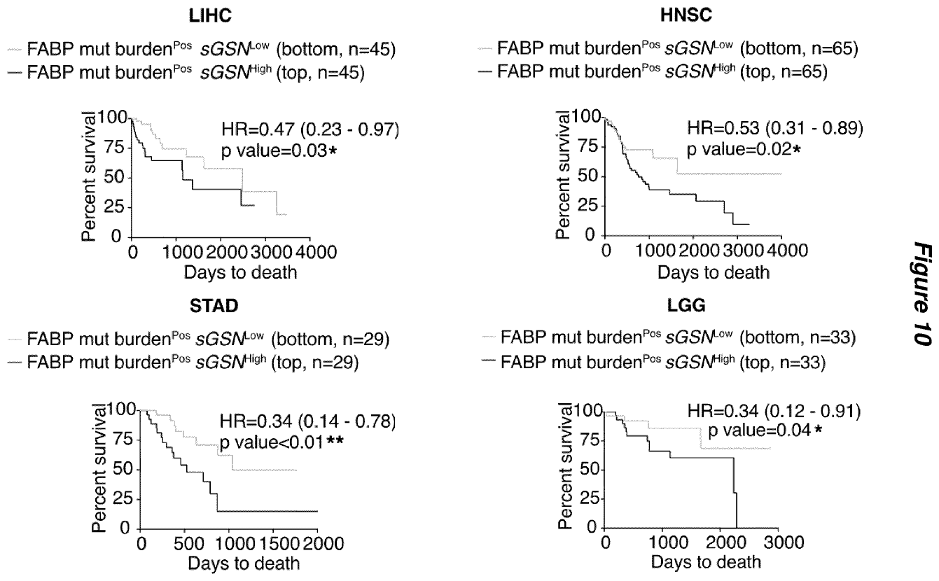

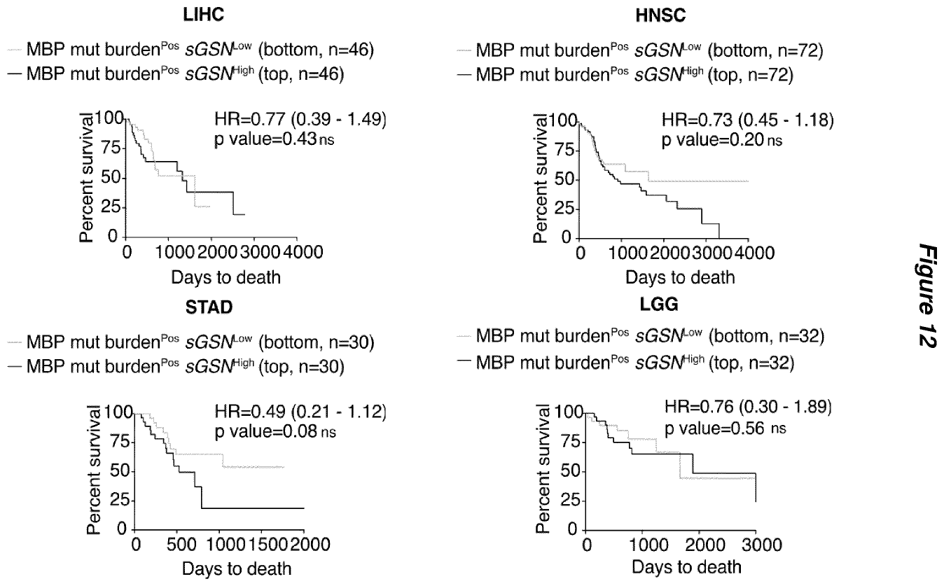

DNGR-1-dependent control of cancer in sGsn^{-/-} mice was most marked for transplantable tumours bearing the LA-OVA model antigen. This suggested that neoantigens resulting from mutations in proteins that associate with F-actin might be preferentially immunogenic in sGSN^{Low} patients. The inventors therefore examined LIHC, HNSC and STAD patients for mutational burden in F-actin binding proteins (FABPs) compared to total mutational burden or, as a control, mutational burden in microtubule-binding proteins (MBPs). In LIHC, HNSC and, in particular, in STAD the inventors identified multiple patients that had one or more mutations in the coding regions of one or more genes encoding FABPs (FIG. 9). LIHC but not HNSC and STAD patients bearing FABP mutations displayed better overall survival in the absence of additional stratification. However, when patients were further stratified by intratumoural sGSN transcript levels, it became obvious that the combination of low sGSN together with mutations in FABP offered the best correlation with overall survival across all three cancers (FIG. 10, 11). This effect of low intratumoural sGSN on overall survival was not seen when the analysis was performed with (cytoplasmic) cGSN transcripts as variable and was specific to patients with mutations in FABPs as it was not seen with stratification on the basis of total mutational burden or mutations in MBPs (FIG. 12). Moreover, even in a cancer, such as low grade glioma (LGG, n=515), where low sGSN expression did not by itself predict survival, mutational burden in FABPs but not total mutational burden or mutations in MBPs revealed a correlation with survival of intratumoural sGSN expression. As seen with STAD patients, increased patient survival in LGG required the intersection of low levels of sGSN transcripts and high levels of CLEC9A message. In the sub-group of LGG with lower sGSN expression, there was a strong correlation between gene signatures for CLEC9A+“ER phagosome pathway” and “effector CD8 T cell”+“cross-presentation related”. Collectively, these data suggest that low sGSN expression may selectively enhance immune responses to neoantigens associated with the actin cytoskeleton even in cancers such as LGG and LIHC with minimal to low mutational burden, thereby increasing cancer patient survival.

Cross-priming of CD8+ T cells against tumour antigens is a cornerstone of anti-tumour immunity. Type 1 conventional dendritic cells are key players in this process and the abundance of the CLEC9A cDC1 hallmark transcript in tumours correlates positively with cancer patient survival³³⁻³⁶. However, whether this reflects a role for the CLEC9A gene product, DNGR-1, in anti-tumour immunity has remained unclear.

Here, the inventors show that DNGR-1 can promote anti-tumour immunity but this effect is masked by sGSN either produced locally in the tumour microenvironment or derived from plasma. Thus, sGSN can dictate the degree to which tumour antigenicity is revealed to the CD8⁺ T cell compartment via cDC1 DNGR-1-mediated cross-presentation. The inventors work further suggest that the latter favours priming of anti-tumour CD8⁺ T cells specific for mutated proteins that are part of the actin cytoskeleton. Interestingly, the inventors found that mutations in F-actin binding proteins occur frequently in the vast majority of human cancers, and can generate tumour neoantigens in both mice and humans^{46,47}. The fact that such mutations correlate with better prognosis specifically in patients whose tumours have relatively low sGSN transcript levels suggests that local production of sGSN in the tumour microenvironment may be a means of evading DNGR-1-dependent induction of anti-tumour immunity. For other cancers, circulating levels of sGSN in plasma might be sufficient to dampen anti-tumour immunity, which could help explain the lower prevalence of fatal cancers in patients with Meretoja's disease, in which proteolytic cleavage causes loss of sGSN function⁴⁸. Conversely, whether sGSN inhibition of DNGR-1 activity normally helps prevent inappropriate immune responses to cytoskeletal antigens (e.g., in myositis) remains to be assessed although we note that sGsn^{-/-} mice do not display signs of overt autoimmunity. Transiently targeting the interaction between sGSN and F-actin might therefore be a safe and attractive therapeutic strategy to boost the antigenic visibility of tumour cells, which could show promise in conjunction with checkpoint blockade immunotherapy for augmenting CD8⁺ T cell-mediated cancer control even in patients with low mutational burden.

### Methods

### Mice

Mice selectively lacking sGSN (sGsn^{-/-} ) were generated by microinjection of mRNA Cas9(D10A) and in vitro transcribed paired guide RNAs (gRNAs), targeting the alternatively spliced exon coding for the signal peptide of the sGSN gene product, into fertilised single cell staged C57BL/6J embryos. Embryos carrying correctly targeted mutations were selected and founder lines were established. One founder line carrying a targeted allele was designated Gsn^{em2(sGsn)Crs} and used for these studies. Gc^{-/-} mice carrying the Gc^{tm1.1(KOMP)Vlcg} allele on a C57BL/6 background were purchased from KOMP repository. Mice doubly deficient for either sGSN and DNGR-1 (sGsn^{-/-};Clec9a^{gfp/gfp}) or sGSN and Gc (sGsn^{-/-} ;Gc^{-/-}) were generated by crossing sGsn^{-/-} mice with either DNGR-1-deficient mice (Clec9a^{tm1.1Crs} a.k.a., Clec9^{agfp/gfp 21}) or Gcs^{-/-} mice (all on a C57BL/6 background). The above mice, as well as C57BL/6, Clec9a^{gfp/gfp}, another line of DNGR-1 deficient mice Clec9a^{cre/cre}; ³⁷) and OT-I x Rag1^{-/-} mice were bred at the animal facility of the Francis Crick Institute. Mouse genotypes were determined using real time PCR with specific probes designed for each gene (Transnetyx, Cordova, TN). Serum was collected from aged C57BL/6J and sGsn^{-/-} mice, and sent to the UT Southwestern Medical Centre Microarray Core facility for autoantibody determination using their autoantigen microarray. Mice were used at 5-12 weeks of age. For tumour experiments mice were sex-matched and littermates of the same sex were randomly assigned to treatment or control groups. Animal experiments were performed in accordance with national and institutional guidelines for animal care and were approved by the Francis Crick Institute Biological Resources Facility Strategic Oversight Committee (incorporating the Animal Welfare and Ethical Review Body) and by the Home Office, UK.

### Reagents

Purified, labelled (biotin, rhodamine) and unlabelled, rabbit muscle actin proteins and ABPs including purified rabbit muscle myosin II as well as recombinant human plasma GSN and cofilin were from Cytoskeleton Inc. 2 μm non-fluorescent SA-coated microbeads were from Polysciences. FLAG-tagged recombinant dimeric mDNGR-1 ECD was prepared as described 1. OVA-peptide (SIINFEKL) was generated by the peptide chemistry science and technology platform (STP) of the Francis Crick Institute. Ovalbumin (OVA) was either from Calbiochem or a crude preparation of hen egg white³⁸. R-PE-conjugated H-2Kb/SIINFEKL pentamer was from Proimmune. Poly(I:C) was from Invivogen. Mouse serum was prepared from blood collected by cardiac puncture, immediately placed into clotting-activator containing microtubes (1.1 ml Z-gel, Sarstedt), allowed to coagulate for 30 min at room temperature and centrifuged (10,000 rpm, 2 min). Serum-containing supernatant was used after heat-inactivation (56° C., 30 min) or untreated as indicated.

### Cells

5555 Braf^{V600E} and MCA-205 tumour cells as well as different OVA-expressing tumour lines (EG-7, B16-LA-OVA-mCherry and B16-OVA-GFP, MCA-205-LA-OVA-mCherry) were used or tumour growth profile studies in vivo. Tumour cell lines, bm1OVAMEF and BWZ cells were grown in RPMI 1640 containing 10% FCS, 2 mM glutamine, 50 μM 2-mercaptoethanol, 100 units/ml penicillin, 100 μg/ml streptomycin (R10). BWZ cells are stably transduced with mouse CLEC9A fused with the z-chain of the T cell receptor and express a β-gal reporter for nuclear factor of activated T cells (NFAT) 21. 5555Braf^{V600E} cells stably knocked down for cytoplasmic The MutuDC1940 line³⁹ was a kind gift from Hans Acha-Orbea and was cultured in IMDM medium containing 10% FCS, 50 μM 2-mercaptoethanol, 100 units/ml penicillin, 100 μg/ml streptomycin. All media and media supplements were from Life Technologies except for FCS (Source Bioscience).

### Retro Viral and Lentiviral Transduction

For retroviral transduction of tumour cell lines, retrovirus was packaged in 293T cells transfected with a mixture of plasmids: 2 μg of pVSV-G envelope protein-coding plasmid, 3.72 μg of pHIV (gag-pol) packaging plasmid and 10 μg of pMSCV-IRES-Life-Act-OVA-mCherry plasmid using Lipofectamine 2000 (Invitrogen). After two days post-transfection, the pseudotyped virus-containing culture media was harvested, filtered and used to infect target cells (B16F10 and MCA-205) in the presence of 10 μg/ml Polybrene. After two rounds of infection the medium in the target cells was exchanged for fresh complete RPMI1640 medium. For positive clone selection the medium was supplemented with puromycin (1.5 μg/ml for B16F10 and 5 μg/ml for MCA-205) and after three passages target cells were FACS-sorted based on mCherry expression. For lentiviral transduction, 293T cells were co-transfected with a mixture of 2 μg of pVSV-G envelope protein-coding plasmid, 3.72 μg of psPAX2 packaging plasmid and 10 μg of PLKO.1-puro-GsnShRNA (mouse shRNA, TRCN0000071930, mature sequence anti-sense: TTCAGACACGTGTACTTGAGC) using Lipofectamine 2000 (Invitrogen). Viral infection and subsequent selection was performed as above. 5555 Braf^{V600E} Gsn knockdown (KD) cells were positively selected using puromycin (1 μg/ml) containing medium.

### Tumour Cell Injections

Tumour cells were dissociated with trypsin (0.25%), and washed three times in PBS. The final cell pellet was resuspended and diluted in endotoxin-free PBS (between 0.2×10⁶ to 0.5×10⁶ cells per 100 μl) and injected s.c. in the shaved right flank of each recipient mouse. Tumour growth was monitored every 1 to 3 days, and the longest tumour diameter (l) and perpendicular width (w) were measured using digital Vernier callipers; tumour volume was calculated using the formula: length×width²/2.

In vivo Administration of Immune-Checkpoint Blockade Therapy

For immune-checkpoint therapy in vivo, anti-PD1 monoclonal antibody (clone RMP1-14, BioXCell, BE0146) or rat IgG2a isotype control (clone 2A3, BioXCell, BE0089) was administered i.p. at 200 μg/200 μl PBS per mouse from day 3 post-tumor cell transplantation, every 3 days for a maximum of six doses. For the combination therapy of poly(I:C) with anti-CTLA-4, mice received 50 μg/50 μl of poly(I:C) (VacciGrade, InvivoGen, vac-pic) or 50 μL of PBS injected intratumorally on days 7 and 11 post-tumor cell transplantation, and either anti-CTLA-4 monoclonal antibody (clone 9D9, BioXCell, BP0164) or rat IgG2b isotype control (clone MPC-11, BioXCell BE0086) 50 μg/200 μl i.p. on days 6 and 12.

In vivo CD8 T Cell Depletion

For CD8⁺ T cell depletion, mice received 300 μg/200 μl of anti-CD8 (clone 2.43, BioXCell, BE0061) or rat IgG2b isotype control (clone LTF-2, BioXCell, BE0090) i.p. from 3 days prior to inoculation of tumour cells and followed twice per week until the end of the experiment (days: 1, 4, 7, 10, 13).

### Dot Blot Binding Assay and Western Blot

Binding of DNGR-1 to in vitro polymerised F-actin was analysed by dot blot as described previously^{1,3}. Briefly, F-actin was transferred onto nitrocellulose membranes by gravity flow using a dot blot apparatus. Post-transfer, NC membranes were blocked in 5% milk, cut into strips, and either probed directly as per the published protocol or incubated with mouse serum, FCS or the purified ABPs in blocking solution (5% milk) for 1-2hrs, washed and then probed with FLAG-tagged mDNGR-1 ECD followed by HRP-conjugated mouse anti-FLAG antibody (M2, Sigma, 1:20,000 dilution).

For Western blot of mouse serum, equivalent volumes of serum samples were diluted in Laemmli buffer, resolved using reducing SDS-PAGE and transferred to nitrocellulose membranes (Merck-Millipore). For cytoplasmic gelsolin, splenic lysates were prepared by homogenization using a TissueLyser II (QIAGEN) in cold protein lysis buffer (RIPA supplemented with protease inhibitors (Roche) before quantification of supernatants using BCA (Thermo Fisher Scientific). Equal volumes of protein were diluted in Laemmli buffer, resolved using reducing SDS-PAGE and transferred to nitrocellulose membranes (Merck-Millipore). Secreted, cytoplasmic gelsolin and OVA levels were assessed by probing membranes with anti-gelsolin antibody (D9W8Y, Cell Signaling Technology, 1:1000 dilution) anti-OVA antibody (polyclonal antibody, Sigma, 1:1000) respectively followed by HRP-anti-rabbit antibody (1:5000 dilution). Loading controls for serum and splenic lysates were assessed using the following antibodies, respectively: mouse IgG light chain, HRP-β-actin (AC-15, Sigma, 1:10,000). Visualization was carried out with the SuperSignal West Pico Chemiluminescent substrate kit (Thermo Fisher Scientific).

### Preparation of F-Actin

F-actin was prepared as described^{1,3}. Briefly, G-actin (10 mg/ml, 200 μM) stock was diluted 1:10 in a mixture of 1× G-actin buffer and 10× F-actin buffer and left at RT for at least 1 hr to induce filament formation. Soluble F-actin (20 μM) was then diluted 1:4 in PBS. F-actin was incubated for 1 hr at RT and adjusted to the final assay concentration (top dose) with PBS. Dilution series of F-actin preparations were prepared in PBS and used directly for dot blot and reporter cell assays. For coupling to beads, biotinylated, fluorescent F-actin was prepared by mixing equal amounts (20 μl) of rhodamine-G-actin and biotinylated G-actin (both at 20 μM, 1 mg/ml) in the presence of equimolar concentration (20 μM) of phalloidin in 5 μl G-buffer followed by addition of 5 μl 10×F-buffer to start the polymerisation reaction (1 hr, RT). 12.5 μl of phalloidin-stabilised, rhodamine-labeled and biotinylated F-actin (16 μM) was mixed with 37.5 μl PBS (for F-actin beads) for a final concentration of 4 μM and incubated for 1 hr at RT.

Gelsolin Treatment of F-Actin, F-Actin Coupled to Microspheres

4 μM biotin/rhodamine-F-actin or biotin/rhodamine-F-actin was diluted 1:4 with PBS and 100 μl was added to 20 μl streptavidin-coated beads (2 μm; Polysciences Inc.), which had been washed twice with wash buffer (PBS+1% BSA), for 30 min on ice. Washed beads were resuspended in wash buffer and sonicated (2×2min) in a water bath sonicator before storage.

F-actin-coupled microbeads were resuspended in HBSS containing 1 mM Ca²⁺ and 10 μg/ml sGsn and incubated for 30 min on ice, followed by addition of FLAG-mDNGR-1 reagent. Beads were washed and stained with fluorescent-labelled antibodies including PE-conjugated rat-anti-DNGR-1 antibody (1F6), AlexaFluor488-conjugated mouse-anti-human gelsolin antibody and mouse anti-actin antibody (AC-40).

In vitro Cross-Presentation

cDC1-mediated cross-presentation of bm1OVAMEF and 5555Braf^{V600E} cells was carried out as described recently³. Briefly, cells were UV-irradiated (240 mJ/cm2) and left for several hours in serum-free RPMI1640 medium. 5555BrafV600E cells were additionally pulsed with OVA (10 mg/ml) for 1 hr at 37° C. Dead cells were added to Mutu DCs (1×10⁵/well) at the indicated ratio and cultured in 96-well round-bottom plates at 37° C. in RPMI 1640 medium containing 2 mM glutamine, 50 μM 2-mercaptoethanol, 100 units/ml penicillin, 100 μg/ml streptomycin and 2.5% heat-inactivated sGsn-deficient mouse serum. To facilitate dead cell uptake, plates were centrifuged at 1000 rpm for 3 min at the start of the incubation. Pre-activated OT-I T cells (5×10⁴/well)⁴⁰ were added after 4 hr and OT-I T cell activation was determined by measuring IFNg levels in the supernatant of overnight cultures by ELISA.

N. brasiliensis Infection Model

N. brasiliensis was obtained as faecal cultures from the lab of Judy Allen (University of Manchester). L3 larvae were extracted by use of a modified Baermann apparatus and collected in PBS. After at least 3 rounds of washing in sterile PBS, larval numbers were counted and further diluted as needed. Mice were infected subcutaneously with 250 L3 larvae per mouse.

### RT-qPCR

Lungs were harvested and bronchio-alveolar lavage fluid (BALF) samples were immediately transferred into lysis buffer. Approximately 20 μg lung tissue were taken from each lung, homogenised using a TissueLyser II (Qiagen) and clarified using QiaShredder columns (Qiagen). RNA was extracted using a column-based method (Qiagen). cDNA synthesis was performed using SuperScript II Reverse Transcriptase (Thermo Fisher Scientific), and random hexamers (Thermo Fischer Scientific). cDNA was then diluted eight times in nuclease-free water and analysed for gene expression by qPCR using PowerUp SYBR Green master mix (Thermo Fisher Scientific). Reactions were carried out using QuantStudio 3 or QuantStudio 5 machines (Thermo Fisher Scientific). Relative expression values were calculated from ΔCts using 18S mRNA as a reference gene. BALF samples were centrifuged for 8 min at 1400 rpm and the supernatant stored at −80° C. until further use. The pellet was resuspended in FACS buffer (PBS with 4% FCS, 5 mM EDTA and 0.2% azide), washed once, and then resuspended in PBS for staining.

### Flow Cytometry of Processed Tumour Tissue, Tumour Draining Lymph Nodes and Non-Draining Primary and Secondary Lymphoid Organs

Tumors and tumour draining lymph nodes (tdLN) were excised at the indicated days after transplantation. Tumor mass of individual tumors was determined using a microscale. For subsequent analysis by flow cytometry, tumors and tdLN were cut into pieces and digested with collagenase IV (200 U/ml) and DNase I (100 μg/ml) for 30 min at 37° C. Tissue was passed through a 70 μm cell strainer (Falcon), washed with FACS buffer (PBS with 1% FCS and 2 mM EDTA) and cells were incubated with Fc block (CD16/32, clone 2.4G2, BD Biosciences) for 10 min in 4° C. before proceeding with antibody mediated staining.

For the ex vivo analysis of T cells, cell suspensions were stained with PE-conjugated H-2K^{b}/SIINFEKL pentamer (Prolmmune) for 15 min at RT. Cells washed and stained with LIVE/DEAD™ Fixable Blue Dead Cell dye (ThermoFischer Scientific) according to manufacturer's protocol and subsequently stained with various lineage specific antibodies: V500-CD45 (30-F11, BD Biosciences, 1:100 dilution), APC-CD3e (145-2C11, 1:100 dilution), APC-Cy7-CD8α (53-6.7, Biolegend, 1:200 dilution). Cells were fixed prior to analysis.

For the ex vivo analysis of cDC1 cells and for phenotypic characterization of sGsn^{-/-} mice, cells from primary and secondary lymphoid tissues were digested as before and stained with LIVE/DEAD™ Fixable Blue Dead Cell dye (ThermoFischer Scientific) according to manufacturer's protocol and subsequently stained in the presence of various lineage specific antibodies: BV421-XCR-1 (ZET, Biolegend, 1:100 dilution), BV605-Ly6C (HK1.4, Biolegend, 1:100 dilution), BV605-CD8α (53-6.7, Biolegend, 1:200 dilution), BV605-CD45.2 (104, Biolegend, 1:200 dilution), BV650-B220/CD45R (RA3-6B2, Biolegend, 1:200 dilution), BV711-CD45.2 (104, Biolegend, 1:200 dilution), FITC-CD11b (M1/70, BD Biosciences, 1:100 dilution), PerCP-Cy5.5-GR-1 (RB6-8C5, Biolegend, 1:100 dilution), PerCP-Cy5.5-CD4 (RM4-5, BD Biosciences, 1:200 dilution), PerCP-Cy5.5-CD103 (2E7, Biolegend, 1:100 dilution), PE-NK1.1 (PK136, BD Biosciences, 1:100 dilution), PE-CD4 (RM4-5, BD Biosciences, 1:200 dilution), PE-Cy7-TCR-delta (GL3, Biolegend, 1:100 dilution), PE-Cy7-CD64 (X54-5/7., Biolegend, 1:100 dilution), AlexaFluor647-Sirp-1α (P84, Biolegend, 1:100), APC-CD8α (53-6.7, BD Biosciences, 1:200 dilution), AlexaFluor700-MHC-II(I-A/I-E) (M5/114.15.2, E-Bioscience, 1:100 dilution), APCeFluor780-CD11c (N418, E-Bioscience, 1:100 dilution), APC-Cy7-TCRbeta (H57-597, Biolegend, 1:200 dilution). Cells were fixed prior to analysis.

Fixation were perfomed using the Fixation/Permeabilisation buffer-Foxp3 Kit (E-Biosciences) according to the manufacturer's protocol. Samples were acquired on a Fortessa X20 B (BD Biosciences). Data were analysed using FlowJo software. All the information for the antibodies used can be found in Table S6.

For the immunophenotypic characterisation of primary and secondary lymphoid organs immune populations have been defined as follow: B cells (live CD45.2⁺ CD3⁻ CD19⁺), NK cells (live CD45.2⁺ CD3⁻ CD19⁺ NK1.1⁺), T cells (live CD45.2⁺ CD19⁻ CD3⁺ NK1.1⁻), NKT cells (live CD45.2⁺ CD19⁻ CD3⁺ NK1.1⁺). TCR gamma delta T cells (live CD45.2⁺ CD19⁻ CD3⁺ NK1.1⁻ TCR delta⁺). TCR alpha beta T cells (live CD45.2⁺ CD19⁻ CD3⁺ NK1.1⁺ TCR beta⁺), TCR alpha beta cells were further subdivided to helper (CD4⁺) and cytotoxic (CD8⁺) T cells, thymic double positive (DP) T cells (live CD45.2⁺ CD4⁺ CD8⁺), macrophages (live CD45.2⁺ CD11cMHCII douple positive⁻ CD11b⁺ CD64⁺ GR-1⁻ Ly6C⁻, neutrophils (live CD45.2⁺ CD11cMHCII douple positive⁻ CD11b⁺ CD64⁺ GR-1^{High} Ly6C^{Low}, monocytes (live CD45.2⁺ CD11cMHCII douple positive⁻ CD11b⁺ CD64⁺ GR-1^{Low} Ly6C^{High}). resident cDC (live CD45.2⁺ CD64⁻ B220⁺ CD11c⁺ MHCII^{Low}), and migratory cDC (live CD45.2⁺ CD64⁻ B220⁻ CD11c⁺ MHCII^{High}), res./migr. cDC were further subdivided in cDC1 (XCR-1⁺ CD11b⁻) and cDC2 (XCR⁻ CD11b⁺, pDC(live CD45.2⁺ CD64⁻ B220⁺ CD11c⁺ MHCII^{Low}).

### NFAT Reporter Assay in BWZ Cells

For measuring the effect of sGSN on the agonistic activity of F-actin, myosin modified F-actin or dead cells, the inventors used an NFAT reporter assay as described previously^{1,21}. Briefly, BWZ-mDNGR-1-z-chain cells were plated in 96 well plates (1×105 cells/well) in the presence of added stimuli as indicated. Stimulation of reporter cells was performed in RPMI 1640 medium containing 2 mM glutamine, 50 μM 2-mercaptoethanol, 100 units/ml penicillin, 100 μg/ml streptomycin and 2.5% sGsn-deficient mouse serum. After overnight culture, cells were washed once in PBS and LacZ activity was measured by lysing cells in CPRG (Roche)-containing buffer. 1-4 hours later O.D. 595 was measured using O.D. 655 as a reference.

### Bioinformatic Analysis of Cancer Patient Data

Normalised read counts for gelsolin isoform expression were downloaded from the Genotype-Tissue Expression (GTEx) resourse Biobank [gtexportal website]. Raw count data for each TCGA dataset was downloaded from https://gdac.broadinstitute.org/ and normalised using DESEQ2⁴¹. Tumour only samples were ranked using normalised GSN expression. Differential 461 expression between Low and High expressing GSN groups was determined using the Wald's test. The Wald's statistic was used to rank genes using Preranked GSEA (version 2.2.3)⁴² and statistically significant pathways identified from the c2 pathway genesets [MSigdb]43. Overall survival analyses were performed for the high and low expression ranked values for cytoplasmic (cGSN:uc011lyh and uc010mvu) and secreted (sGSN: uc004ble) GSN isoforms and plotted for Kaplan-Meier curves using GraphPad Prism (GraphPad). using the REACTOME database. MHC class I (cross)-presentation, cell death and immunity gene signatures can be found in REACTOME pathway database [reactome website]. cDC1 gene signature is composed of the following genes: CLEC9A, XCR1, CKNK, BATF3³⁴. Effector CD8 T cell gene signature is composed of the following genes: CD3, CD8A, CXCL10, CXCL9, GZMA, GZMB, IFNG, PRF1^{34,45}. Total tumour mutational counts, mutational counts for F-actin binding proteins and microtubule binding proteins for each TCGA dataset were downloaded from the TCGA Pan-Cancer Atlas [cbioportal website].

### Statistical Analysis

All statistical analyses were performed using GraphPad Prism software (GraphPad). Statistical significance was determined using an unpaired two-tailed Student's t test. Statistical analyses for three or more groups and tumor growth profiles were done by ANOVA. The Log-rank (Mantel-Cox) test was used to determine statistical significance for overall survival in cancer patient data from TCGA. In the gene-enrichment analysis using genes were ranked by the Wald's test false discovery rate (FDR)-adjusted p were calculated. Auto-antibody scores were compared using two-tailed Wilcoxon matched-pairs signed rank test. Pearson's correlation coefficient (r) was calculated as a measure of the strength of the association between the expression values between of two genes or gene signatures. Finally, two-tailed chi-square was used to determine any significant differences in frequencies of different clinical parameters between two groups. Data are shown as mean±s.d. or mean±s.e.m. as indicated in the figure legends. Significance was assumed with *p<0.05; **p<0.01; ***p<0.001, ****p<0.0001.

### References

A number of publications are cited above in order to more fully describe and disclose the invention and the state of the art to which the invention pertains. Full citations for these references are provided below. The entirety of each of these references is incorporated herein.
1. Ahrens, S. et al. F-actin is an evolutionarily conserved damage-associated molecular pattern recognized by DNGR-1, a receptor for dead cells. Immunity 36, 635-645 (2012).
2. Hanč, P. et al. Structure of the Complex of F-Actin and DNGR-1, a C-Type Lectin Receptor Involved in Dendritic Cell Cross-Presentation of Dead Cell-Associated Antigens. Immunity 42, 839-849 (2015).
3. Schulz, O. et al. Myosin II Synergizes with F-Actin to Promote DNGR-1-Dependent Cross-Presentation of Dead Cell-Associated Antigens. Cell Rep 24, 419-428 (2018).
4. Stossel, T. P. et al. Nonmuscle actin-binding proteins. Annu. Rev. Cell Biol. 1, 353-402 (1985).
5. Hartwig, J. H. & Kwiatkowski, D. J. Actin-binding proteins. Curr. Opin. Cell Biol. 3,87-97 (1991).
6. T D Pollard, A. & Cooper, J. A. Actin and Actin-Binding Proteins. A Critical Evaluation of Mechanisms and Functions. Annu. Rev. Cell Biochem. 55, 987-1035 (2003).
7. Lee, W. M. & Galbraith, R. M. The extracellular actin-scavenger system and actin toxicity. N. Engl. J. Med. 326, 1335-1341 (1992).
8. Lind, S. E., Smith, D. B., Janmey, P. A. & Stossel, T. P. Role of plasma gelsolin and the vitamin D-binding protein in clearing actin from the circulation. J. Clin. Invest. 78, 736-742 (1986).
9. Haddad, J. G., Harper, K. D., Guoth, M., Pietra, G. G. & Sanger, J. W. Angiopathic consequences of saturating the plasma scavenger system for actin. PNAS 87, 1381-1385 (1990).
10. Vasconcellos, C. A. & Lind, S. E. Coordinated inhibition of actin-induced platelet aggregation by plasma gelsolin and vitamin D-binding protein. Blood 82, 3648-3657 (1993).
11. Meier, U., Gressner, O., Lammert, F. & Gressner, A. M. Gc-globulin: roles in response to injury. Clin. Chem. 52, 1247-1253 (2006).
12. Moon, A. & Drubin, D. G. The ADF/cofilin proteins: stimulus-responsive modulators of actin dynamics. Mol. Biol. Cell 6, 1423-1431 (1995).
13. Carlier, M. F., Ressad, F. & Pantaloni, D. Control of actin dynamics in cell motility. Role of ADF/cofilin. J. Biol. Chem. 274, 33827-33830 (1999).
14. Sun, H. Q., Yamamoto, M., Mejillano, M. & Yin, H. L. Gelsolin, a multifunctional actin regulatory protein. J. Biol. Chem. 274, 33179-33182 (1999).
15. Kwiatkowski, D. J. Functions of gelsolin: motility, signaling, apoptosis, cancer. Curr. Opin. Cell Biol. 11, 103-108 (1999).
16. Kwiatkowski, D. J., Mehl, R., Izumo, S., Nadal-Ginard, B. & Yin, H. L. Muscle is the major source of plasma gelsolin. J. Biol. Chem. 263, 8239-8243 (1988).
17. Kwiatkowski, D. J. et al. Plasma and cytoplasmic gelsolins are encoded by a single gene and contain a duplicated actin-binding domain. Nature 323, 455-458 (1986).
18. Kwiatkowski, D. J., Mehl, R. & Yin, H. L. Genomic organization and biosynthesis of secreted and cytoplasmic forms of gelsolin. J. Cell Biol. 106, 375-384 (1988).
19. Witke, W. et al. Hemostatic, inflammatory, and fibroblast responses are blunted in mice lacking gelsolin. Cell 81, 41-51 (1995).
20. Cantù, C. et al. Defective erythroid maturation in gelsolin mutant mice. Haematologica 97, 980-988 (2012).
21. Sancho, D. et al. Identification of a dendritic cell receptor that couples sensing of necrosis to immunity. Nature 458, 899-903 (2009).
22. Lees, A., Haddad, J. G. & Lin, S. Brevin and vitamin D binding protein: comparison of the effects of two serum proteins on actin assembly and disassembly. Biochemistry 23, 3038-3047 (1984).
23. Haddad, J. G. et al. Identification of the sterol- and actin-binding domains of plasma vitamin D binding protein (Gc-globulin). Biochemistry 31, 7174-7181 (1992).
24. Cooke, N. E. & Haddad, J. G. Vitamin D binding protein (Gc-globulin). Endocr. Rev. 10, 294-307 (1989).
25. Böttcher, J. P. & Reis e Sousa, C. The Role of Type 1 Conventional Dendritic Cells in Cancer Immunity. Trends Cancer 4, 784-792 (2018).
26. Wculek, S. K. et al. Dendritic cells in cancer immunology and immunotherapy. Nature Reviews Immunology 2012 12:8 20, 7-24 (2020).
27. Galluzzi, L., Buqué, A., Kepp, O., Zitvogel, L. & Kroemer, G. Immunogenic cell death in cancer and infectious disease. Nature Reviews Immunology 2012 12:8 17, 97-111 (2017).
28. Yatim, N., Cullen, S. & Albert, M. L. Dying cells actively regulate adaptive immune responses. Nature Reviews Immunology 2012 12:8 17, 262-275 (2017).
29. Matsushita, H. et al. Cancer exome analysis reveals a T-cell-dependent mechanism of cancer immunoediting. Nature 482, 400-404 (2012).
30. Riedl, J. et al. Lifeact: a versatile marker to visualize F-actin. Nat. Methods 5, 605-607 (2008).
31. Zelenay, S. et al. The dendritic cell receptor DNGR-1 controls endocytic handling of necrotic cell antigens to favor cross-priming of CTLs in virus-infected mice. J. Clin. Invest. 122, 1615-1627 (2012).
32. Chen, C.-C. et al. Secreted gelsolin desensitizes and induces apoptosis of infiltrated lymphocytes in prostate cancer. Oncotarget 8, 77152-77167 (2017).
33. Barry, K. C. et al. A natural killer-dendritic cell axis defines checkpoint therapyresponsive tumor microenvironments. Nat. Med. 24, 1178-1191 (2018).
34. Böttcher, J. P. et al. NK Cells Stimulate Recruitment of cDC1 into the Tumor Microenvironment Promoting Cancer Immune Control. Cell 172, 1022-1037.e14 (2018).
35. Michea, P. et al. Adjustment of dendritic cells to the breast-cancer microenvironment is subset specific. Nat. Immunol. 19, 885-897 (2018).
36. Broz, M. L. et al. Dissecting the tumor myeloid compartment reveals rare activating antigen-presenting cells critical for T cell immunity. Cancer Cell 26, 638-652 (2014).
37. Schraml, B. U. et al. Genetic tracing via DNGR-1 expression history defines dendritic cells as a hematopoietic lineage. Cell 154, 843-858 (2013).
38. Boes, M. et al. T cells induce extended class II MHC compartments in dendritic cells in a Toll-like receptor-dependent manner. J. Immunol. 171, 4081-4088 (2003).
39. Fuertes Marraco, S. A. et al. Novel Murine Dendritic Cell Lines: A Powerful Auxiliary Tool for Dendritic Cell Research. Front. Immunol. 3, (2012).
40. Hanč, P. et al. A pH- and ionic strength-dependent conformational change in the neck region regulates DNGR-1 function in dendritic cells. EMBO J. 35, 2484-2497 (2016).
41. Love, M. I., Huber, W. & Anders, S. Moderated estimation of fold change and dispersion for RNA-seq data with DESeq2. Genome Biol. 15, 550-21 (2014).
42. Subramanian, A. et al. Gene set enrichment analysis: a knowledge-based approach for interpreting genome-wide expression profiles. PNAS 102, 15545-15550 (2005).
43. Liberzon, A. et al. Molecular signatures database (MSigDB) 3.0. Bioinformatics 27, 1739-1740 (2011).
44. Stoltenburg, R. et al. SELEX—A (r)evolutionary method to generate high-affinity nucleic acid ligands. Biomolecular Engineering 24; 4, 381-403 (2007)
45. S. Mariathasan et al., TGFβ attenuates tumour response to PD-L1 blockade by contributing to exclusion of T cells. Nature. 554, 544-548 (2018).
46. E. Zorn, T. Hercend, A natural cytotoxic T cell response in a spontaneously regressing human melanoma targets a neoantigen resulting from a somatic point mutation. European Journal of Immunology. 29, 592-601 (1999).
47. H. Matsushita et al., Cancer exome analysis reveals a T-cell-dependent mechanism of cancer immunoediting. Nature. 482, 400-404 (2012).
48. E.-K. Schmidt et al., Causes of death and life span in Finnish gelsolin amyloidosis. Ann. Med. 48, 352-358 (2016).

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method of treating a cancer in a subject, the method comprising administering a gelsolin inhibitor to the subject to enhance an anticancer immune response, wherein the gelsolin inhibitor inhibits gelsolin from binding F-actin.

2. A gelsolin inhibitor for use in a method of treating cancer, the method comprising administering the gelsolin inhibitor to the subject to enhance an anticancer immune response, wherein the gelsolin inhibitor inhibits gelsolin from binding F-actin.

3. The method according to claim 1, or the gelsolin inhibitor for the use according to claim 2, wherein the gelsolin inhibitor inhibits gelsolin from binding F-actin through a specific binding interaction between the gelsolin inhibitor and gelsolin.

4. The method according to claim 1, or the gelsolin inhibitor for the use according to claim 2, wherein the gelsolin inhibitor inhibits gelsolin from binding F-actin through a specific binding interaction between the gelsolin inhibitor and F-actin.

5. The method, or the gelsolin inhibitor for the use, according to any one of the preceding claims, wherein the gelsolin is secreted gelsolin (sGSN).

6. The method, or the gelsolin inhibitor for the use, according to any one of the preceding claims, wherein the gelsolin is gelsolin that has been released into systemic circulation from a ruptured cell in the tumour microenvironment.

7. The method, or the gelsolin inhibitor for the use, according to any one of the preceding claims, wherein the F-actin is dead cell-associated F-actin.

8. The method, or the gelsolin inhibitor for the use, according to any one of the preceding claims, wherein the F-actin is released into systemic circulation from a ruptured cell in the tumour microenvironment.

9. The method, or the gelsolin inhibitor for the use, according to any one of the preceding claims, wherein the gelsolin inhibitor is an anti-gelsolin antibody.

10. The method, or the gelsolin inhibitor for the use, according to any one of the preceding claims, wherein the treatment comprises the administration of a checkpoint inhibitor to the patient.

11. The method, or the gelsolin inhibitor for the use, according to claim 10, wherein the checkpoint inhibitor is selected from an anti-PD-1, anti-PD-L1, anti-CTLA4, anti-TIM3, anti-KIR, anti-LAG3, and an anti-VISTA antibody.

12. The method, or the gelsolin inhibitor for the use, according to any one of the preceding claims, wherein the cancer is a liver cancer, a head and neck cancer, a glioma, or a gastric cancer.

13. The method, or the gelsolin inhibitor for the use, according to any one of the preceding claims, wherein the treatment comprises the administration of radiotherapy.

14. The method, or the gelsolin inhibitor for the use, according to any one of the preceding claims, wherein the cancer expresses a neoantigen.

15. The method according to claim 14, wherein the neoantigen results from a mutation in an F-actin binding protein (FABP).

16. A method of treating an infectious disease in a subject, the method comprising administering a gelsolin inhibitor to the subject to enhance an immune response against an antigen derived from a disease-causing agent selected from a viral particle, a bacterium, or a parasite, wherein the gelsolin inhibitor inhibits gelsolin from binding F-actin.

17. A gelsolin inhibitor for use in a method of treating an infectious disease in a subject, the method comprising administering the gelsolin inhibitor to the subject to enhance an immune response against an antigen derived from a disease-causing agent selected from a viral particle, a bacterium, or a parasite, wherein the gelsolin inhibitor inhibits gelsolin from binding F-actin.

18. The method according to claim 16, or the gelsolin inhibitor for the use according to claim 17, wherein the gelsolin inhibitor inhibits gelsolin from binding F-actin through a binding interaction between the gelsolin inhibitor and gelsolin.

19. The method according to claim 16, or the gelsolin inhibitor for the use according to claim 17, wherein the gelsolin inhibitor inhibits gelsolin from binding F-actin through a binding interaction between the gelsolin inhibitor and F-actin.

20. The method, or the gelsolin inhibitor for the use, according to any one of claims 16 to 19, wherein the gelsolin is secreted gelsolin (sGSN).

21. The method, or the gelsolin inhibitor for the use, according to any one of claims 16 to 20, wherein the gelsolin is gelsolin that has been released into systemic circulation from a ruptured cell.

22. The method, or the gelsolin inhibitor for the use, according to any one of claims 16 to 21, wherein the F-actin is dead-cell associated F-actin.

23. The method, or the gelsolin inhibitor for the use, according to any one of claims 16 to 22, wherein the F-actin is released into systemic circulation from a ruptured cell.

24. The method, or the gelsolin inhibitor for the use, according to any one of claims 16 to 23, wherein the gelsolin inhibitor is an anti-gelsolin antibody.

25. A method of categorising a cancer patient as having a good prognosis or a bad prognosis, the method comprising measuring the transcript and/or protein expression level of gelsolin in a sample that has been obtained from the patient, and comparing the gelsolin transcript and/or protein expression level against a reference value which is the average level of gelsolin transcript and/or protein expression in samples from a population of other cancer patients of the same disease stage, wherein, if the cancer patient has a lower level of gelsolin transcript and/or protein expression than the reference value, the patient is categorised as having a good prognosis, and if the cancer patient has a higher level of gelsolin transcript and/or protein expression than the reference value, the patient is categorised as having a bad prognosis.

26. The method according to claim 25, wherein the method further comprises measuring the transcript and/or protein expression level of DNGR-1 and/or myosin II in the sample, and comparing these marker transcript and/or protein expression levels against a reference value or reference values which is/are the average level of the respective marker in samples from a population of other cancer patients of the same disease stage.

27. The method according to claim 25 or claim 26, wherein the reference value or values are determined using data from The Cancer Genome Atlas (TCGA).

28. The method according to any one of claims 25 to 27, wherein the cancer expresses a neoantigen.

29. The method according to claim 28, wherein the neoantigen results from a mutation in an F-actin binding protein (FABP).

30. The method according to any one of claims 25 to 29, wherein gelsolin that is measured is secreted gelsolin (sGSN).

31. A method of screening for an immunotherapy agent, the method comprising providing a homogenous population of reporter cells that expresses DNGR-1 and then:
a) contacting one of the reporter cells with F-actin under conditions suitable to detect cross-presentation (XP), to establish a reference level of XP;
b) contacting another of the reporter cells with F-actin in the presence of gelsolin under the same conditions as in a), wherein gelsolin is present at a concentration sufficient to substantially reduce the level of XP; and
c) contacting yet another of the reporter cells with F-actin in the presence of both gelsolin and a candidate agent, wherein the conditions and gelsolin concentration is the same as in b)
wherein, if the level of XP in c) is greater than the level of XP in b), then the candidate agent is selected as an immunotherapy agent.

32. The method of claim 31, wherein the gelsolin is secreted gelsolin (sGSN).

33. Gelsolin for use in a method of treating an autoimmune disease in a subject, the method comprising increasing the level of gelsolin in the subject.

34. The gelsolin for the use according to claim 33, wherein the gelsolin is soluble gelsolin (sGSN).
